(19) United States
(12) Patent Application Publication
(10) Pub. No.: US 2025/0138031 A1
(43) Pub. Date: May 1, 2025

(54) IN VITRO MEASUREMENT OF THE LYSIS OF A FIBRIN CLOT

(71) Applicant: DIAGNOSTICA STAGO, ASNIERES SUR SEINE (FR)

(72) Inventors: Matthieu Daniel Luc BOURDIN, PARIS (FR); Olivier Frédéric Lucien MATHIEU, ERMONT (FR); Charlotte GILES, HOUILLES (FR)

(21) Appl. No.: 18/691,193

(22) PCT Filed: Sep. 14, 2022
(86) PCT No.: PCT/FR2022/051735

§ 371 Date: Mar. 12, 2024

(30) Foreign Application Priority Data
Sep. 14, 2021 (FR) FR2109634

(51) Int. Cl. G01N 33/86; G01N 33/557

(52) U.S. Cl. CPC G01N 33/86; G01N 33/557; G01N 2333/745; G01N 2333/968; G01N 2800/224

(57) ABSTRACT

The invention relates to a method for the in vitro measurement of fibrin clot degradation on the basis of a curve of the lysis of a fibrin clot over time in a blood or plasma sample previously obtained from a patient who may have a deficiency in at least one coagulation factor, said method including determining a basal level value for the fibrin clot at a time t1, and determining a degraded level value for the fibrin clot at a subsequent time t2. The method may comprise an additional step of classifying the tested sample into a group reflecting a lysis profile of a fibrin clot. The invention also relates to a method for monitoring a therapeutic treatment administered to a patient who may have or who does have a deficiency in at least one coagulation factor, said method likewise further comprising a step of adjusting the therapeutic treatment followed by said patient, according to the classification obtained. The invention further relates to the use of an antifibrinolytic agent, in particular tranexamic acid (TXA) or a composition comprising said acid, for use in a therapeutic treatment, said use comprising the carrying out of a method according to invention, and means for implementing the invention.

### TECHNICAL FIELD

This disclosure falls within the field of hemostasis. It concerns more particularly the field of hemophilia, and relates to a method for carrying out an in vitro measurement of a curve of the lysis of a fibrin clot over time in a blood or plasma sample, in order to allow determining the profiles of patients who may be hemophilic, are diagnosed as hemophilic, or are hemophilic, based on the kinetics of the lysis of said clots. According to another aspect, the invention relates to the possibility of adapting a treatment or choosing a treatment based on the physiological parameter observed for these patients, according to the invention.

The invention relates more specifically to a method for the in vitro measurement of fibrin clot degradation on the basis of a curve of the lysis of a fibrin clot over time (kinetic curve) in a blood or plasma sample previously obtained from a patient who may have a deficiency in at least one coagulation factor, as defined in one of claims 1 to 12. This method includes determining a basal level value for the fibrin clot at a time t1, and determining a degraded level value for the fibrin clot at a time t2 that is subsequent to time t1.

According to one aspect, the method may comprise an additional step of classifying the tested sample into a group reflecting a lysis profile of a fibrin clot, as defined in one of claims 13 to 18.

The invention also relates to a method for monitoring a therapeutic treatment administered to a patient who may have or who does have a deficiency in at least one coagulation factor, involving the implementation of a method for carrying out an in vitro measurement of a curve of the lysis of a fibrin clot over time according to the invention, and a classification of the analyzed sample into a group, and making a conclusion as defined in claim 19. Such a method may also further comprise a step of adjusting the therapeutic treatment followed by said patient, according to the classification obtained, as defined in claim 20.

The invention further relates to the use of an antifibrinolytic agent, in particular tranexamic acid (TXA) or a composition comprising said acid, for use in a therapeutic treatment, said use comprising the carrying out of a method according to the invention, as defined in claim 21.

The invention also relates to a screening method as defined in claim 22, and means for implementing the invention, i.e. a data processing system or a device comprising particular means for implementing a method according to the invention, or a computer program, or a non-transitory computer-readable storage medium on which is stored a computer program, or a kit, as defined in claims 23 to 26.

### PRIOR ART

Hemophilia is a recessive congenital disease characterized by spontaneous or prolonged bleeding due to a deficiency in one of the coagulation factors (factors VIII, IX or XI, in the present description also called “FVIII”, “FIX” and “FXI” for “factor VIII”, “factor IX” and “factor XI” respectively). There are three types of hemophilia: Hemophilia A, Hemophilia B, or Hemophilia C. Hemophiliacs are generally classified into three classes according to the factor VIII assay (FVIII assay for hemophilia A, FIX assay for hemophilia B, FXI assay for hemophilia C):
Severe hemophilia (factor is <1%: by convention, 1% of a factor is equivalent to 0.01 IU/mL. The IU/mL unit is also used in this description instead of percentages);
Moderate hemophilia (factors are between 1 and 5%);
Minor hemophilia (factors are between 5% and 40%).

In particular, FVIII deficiency can lead to insufficient fibrin production during coagulation. The clot is thus less resistant and forms with difficulty. The balance between coagulation and lysis is weakened in comparison to a healthy person. An A-hemophiliac will present a higher risk of bleeding in the event of trauma (injury) or spontaneously in the joints and muscles. The more severe the disease, the more frequent the bleeding. The bleeding frequency is assessed by the Annual Bleeding Rate (ABR) which corresponds to the number of bleedings per year.

However, the bleeding risk of hemophiliacs is very heterogeneous: some patients with very low FVIII levels will have a low ABR while patients diagnosed as moderate A-hemophiliacs may bleed more. With FVIII replacement therapy, which is the treatment of choice to date, the goal is to raise the plasma concentration of FVIII to a level that minimizes the time spent below a threshold level (“trough level”). However, this optimal FVIII threshold for bleeding suppression is unpredictable and is not correlated with the patients' clinical phenotype.

It has been highlighted that the hemostatic balance and particularly the process of clot lysis could be modified in hemophilia patients. The association of a low level of FVIII with hyperstimulation of the fibrinolytic system would lead to the predisposition towards bleeding in A-hemophiliacs. Pro-/anti-thrombotic proteins also have an influence (TFPI, Antithrombin, Proteins C and S). These multiple effects cause a difference between the clinical phenotype and the FVIII concentration. Leong and colleagues in 2017 (Leong et al. 2017 Research and practice in thrombosis and haemostasis) indicated that the ability to form an effective hemostatic clot depends on more than just FVIII levels. They raised the possibility that the clinical variation in patient response to FVIII replacement therapy can be reduced, among other things, by modulating the contribution of fibrin, platelets, and erythrocytes to hemostasis. The formation of the fibrin clot and its fibrinolysis, distinctive in hemophilia patients, could influence the severity of the bleeding tendency.

Clinical studies that suggest adapting the dosage of FVIII treatment according to appropriate pharmacokinetic profiles have not achieved zero bleeding. The average ABR rate during prophylaxis (1% FVIII) is 6.3 bleeds/patient/year. The wide ABR range [4,4-9.9] suggests that some patients require a higher dose of FVIII in order to prevent spontaneous bleeding. It is recognized that a level of between 12 and 15% FVIII would maximize bleeding prevention. This rate is difficult to envisage in prophylaxis, due to the short half-life and the cost of treatments.

The Thrombin Generation Test (TGT), the aPTT-based Clot Waveform Analysis (CWA) test, or viscoelastic tests (TEG/ROTEM) have been used in hemophilia A and are recognized as sensitive to variations in FVIII (Chitlur 2012 Thrombosis Research, Challenges in the laboratory analyses of bleeding disorders). The trough level based on the patient's ETP can be more reliable than the 1% FVIII: C trough level (Dargaud 2017) but this is not optimal for monitoring hemophilia-A patients (Tarandowsky 2013). It should be noted that Dargaud and colleagues found that 4 patients among the 11 (i.e. 36%) with the lowest ETPs did not have a bleeding clinical phenotype (Dargaud 2017).

The inventors thus note that the ideal hemostatic test must correlate the biological phenotype of patients with the bleeding clinical phenotype, and allow determining the severity of bleeding. Such characteristics would allow proposing an adaptation of the treatment dosage to patients. To date, no test has been approved for monitoring hemophilia-A patients and for predicting the predisposition of hemophilia patients towards bleeding (Tripodi 2019, Tarandowsky 2013). Indeed, a large inter-patient difference exists in the response to FVIII therapy; the current general tests (TGT, CWA and TEG/ROTEM) are not sufficiently sensitive to small variations in FVIII, particularly in the lower zone (Matsumoto 2009, Aghighi 2019). TGT activated by TF (tissue factor) in itself has a relatively low sensitivity to deficiencies in intrinsic coagulation factors; it is necessary to modify it (diluted TF, CTI, PRP) to achieve sufficient sensitivity. Furthermore, other factors, not explored in these tests, contribute to the severity of the phenotype (Aghighi 2019, Leong 2017, He 2018).

In 2020, three studies by NovoNordisk (Guardian) and three studies by Bayer (Leopold) showed that inter-patient variability in the bleeding phenotype remains significant and unexplained, that the risk of bleeding can change over time and is influenced by factors independent of the FVIII pharmacokinetics and the trough level. The need for a treatment customization tool that takes into account a personalized risk of bleeding remains necessary to this day (Tiede 2020, Abrantes 2019).

In addition to this scientific observation, there are clinical-economic considerations highlighting the utility of a lysis test in hemophiliacs that answers the problems set forth above. In France, the prevalence of hemophilia A is approximately 1/5000 (with 50% of these being severe hemophilia). Treatment with antihemophilic factor corresponds to approximately 98% of the cost of treating a severe hemophiliac. In addition, in France (in 2003), 7,000 hospitalizations per year linked to hemophilia A were counted. Today, the total costs generated by a severe hemophiliac are €800,000 per year on average (Poster P190 ECTH 2018, B. Polack et al.) in France.

In France, according to the HAS 2019 study, 1944 patients are treated prophylactically: for the Advate® molecule: 20 to 40 IU (International Units) of FVIII/kg every 2 to 3 days/Novoeight®: 20 to 40 IU of FVIII/kg every 2 days, up to 50 IU 3 times/week; in case of bleeding, every 8 hours to every 2 days. For Hemlibra®, the target population is 1410 to 1880 patients, once a week or every 2 weeks for 4 weeks or every 4 weeks (HAS 2019 website and National Hemophilia Care Diagnostic Protocol V3 of 2019). By convention, 1 IU=100% of FVIII (or FIX or FXI) as described in the present description.

In India, there are an estimated 170,000 hemophiliacs, of whom 100,000 have the severe form of the disease. With a prophylaxis of 3 IU/person this would mean a required volume of 4 billion IU at 0.25 dollars/IU. The total cost would be 1 billion dollars/year (GFHT COMETH 2019-Symposium “Hemophilia around the world” “Epidemiological data” by Alok Srivastava).

Worldwide, in 2018, the market for hemophilia A treatments was $9.8 billion. This market is constantly increasing, with an estimate of $15.8 billion in 2026 (EAHAD 2020-Symposium “Ethical aspect of gene therapy in children” by Rieke van der Graaf, and “Hemophilia Drug Market” on the Fortune Business Insights website).

These examples illustrate that improving the cost/efficacy balance is a real international issue for the treatment of hemophiliacs and in particular A-hemophiliacs.

The present invention relates, according to one particular embodiment, to a classification of hemophilia patients, based on the kinetics of the lysis of the fibrin clot forming in a sample obtained from these patients, opening up the possibility of selecting or personalizing the maintenance therapy that the patients are receiving. It follows that the present invention opens the way for predicting a treatment adapted to each patient, in the laboratory. For clinicians, the expected advantages are a better characterization of patients by their predisposition to bleeding, on the one hand integrating the variable which consists of their low, moderate, or high bleeding risk, and on the other hand offering a real tool for adapting the treatment. The overall benefits allowed by the invention include reduced treatment costs, improved quality of life for patients, and potentially a reduction in the annual bleeding rate.

Known from WO 2016/012729 is a method for determining the structural profile of a fibrin clot reflecting its stability, for predicting the risk of bleeding, thrombosis, or rethrombosis. However, the disclosed protocol does not provide for the possibility of classifying patients, in particular hemophiliacs, by a statistical or learning method, and the lysis must be interpreted using two wavelengths during the turbidimetric assay performed, which adds to the complexity of implementing the method. Finally, the initial reagents differ.

Furthermore, the state of the art does not report any methodology which allows considering the degradation of a fibrin clot due to its lysis in hemophiliacs, as the only tool to help personalize the treatment of the missing factor type administered to a hemophiliac patient, associated or not associated with antifibrinolytic treatment. By contrast, the present invention allows such an achievement.

### DETAILED DESCRIPTION

This disclosure seeks to address the aforementioned issues.

A method is proposed for the in vitro measurement of fibrin clot degradation based on the curve of the lysis of a fibrin clot over time in a blood or plasma sample previously obtained from a patient who may have or who does have a deficiency in at least one coagulation factor, the method comprising the following steps:
a. Mixing the sample previously obtained from said patient with a composition of reagents comprising tissue factor, phospholipids, tPA (tissue plasminogen activator), and one or more activator(s) of the intrinsic coagulation pathway, in particular one or more of these activator(s) chosen among: ellagic acid, ethylene glycol gallate, silica, FIXa, FXIa, FXIIa, and optionally one or more coagulation factor(s) that are missing in the analyzed patient, in particular chosen among: FVIII, FIX, FXI, a bispecific antibody, or another hemophilia treatment, which can also be used as missing factor(s), or a mixture of two or more of these factors and treatment(s);
b. Incubating the mixture obtained in a., then
c. Triggering coagulation by adding calcium ions to the mixture incubated in step b. in order to allow a fibrin clot to form in the mixture, then allowing lysis of the formed clot, and
d. Measuring the degradation kinetics of the fibrin clot formed and degraded during lysis after step c., and
e. Determining a basal level value of the fibrin clot at a time t1, time t1 being chosen to be located between the Tmax and TL of the curve of the lysis of the fibrin clot, and determining a degraded level value of the fibrin clot at a time t2 that is subsequent to time t1, time t2 being chosen to be between 300 to 900 seconds after t1.

The characteristics of the means employed in this method will be specified below. It will be understood that time t1 is a predetermined time, and that time t2 is also a predetermined time; these two times are fixed prior to executing the measurement method according to the invention.

The features set forth in the following paragraphs may optionally be implemented, independently of each other or in combination with each other.

The Tmax of a lysis curve of a fibrin clot is a value easily determined by those skilled in the art, and in a conventional manner. During formation of a fibrin clot in a sample, which is naturally followed by fibrinolysis (the degradation process of the formed clot), by necessity a curve reflecting the state of the fibrin clot in the sample transitions through a maximum (the moment when the maximum measured value is reached, for example the maximum delta optical density (DOD), within the measurement duration). The time to reach this maximum is denoted Tmax. Conventionally, a curve of fibrin clot generation and lysis starts after the reaction is triggered by the addition of calcium ions (which corresponds to time to). In this patent application, the term “curve of the lysis of a fibrin clot” (or “lysis curve of a fibrin clot”) means the curve measured and/or plotted within the times indicated in the present description, according to any embodiment, including the area corresponding to the end of formation of the fibrin clot up to the area corresponding to total degradation of the fibrin clot. The “curve of the lysis of a fibrin clot” in its entirety is thus synonymous, unless a different interpretation arises from the context, with the “measured curve” or the “plotted curve”.

The TL of a lysis curve of a fibrin clot is also a value easily determined by those skilled in the art, and in a conventional manner. This is the time when the measured value no longer corresponds to more than 50% of the maximum value (which is therefore measured at time Tmax). In other words, this value corresponds to 50% of the maximum value reached when plotting the lysis curve of a fibrin clot.

In this context, time t1 is a time located between Tmax and TL of the lysis curve of the fibrin clot, which can therefore be located either in the plateau (between Tmax and T90% of max) of the curve, or after the plateau (between T90% of max and TL).

According to the invention, time t2 is selected within a fixed range of values after t1.

Thus, according to the invention, times t1 and t2 are predetermined prior to execution of the method for the in vitro measurement of fibrin clot degradation based on a lysis curve of a fibrin clot over time on a sample obtained from a patient.

The above step e. of the method for the in vitro measurement of fibrin clot degradation on the basis of a curve of the lysis of a fibrin clot over time, can therefore alternatively be written: e. determining a basal level value of the fibrin clot at a predetermined time t1, time t1 being chosen to be between Tmax and TL of the curve of the lysis of the fibrin clot, and determining a degraded level value of the fibrin clot at a predetermined time t2 that is subsequent to time t1, time t2 being chosen to be 300 to 900 seconds after t1.

Note that times t1 and t2 are predetermined, meaning they are fixed for the implementation of the method and are independent of the patient whose sample is analyzed, following the instructions in this description. Times t1 and t2 have been previously determined based on a population of patient samples, in order to differentiate said patients as much as possible into different groups reflecting a differing behavior of the clot during lysis, as explained below. Times t1 and t2 and the values respectively measured at these times can thus advantageously serve as classifying parameters once fixed. These new parameters make it possible, as shown below, to obtain information about the analyzed sample, to the patient's benefit.

The optimal choice of times t1 and t2 in the aforementioned ranges can be made by those skilled in the art, by determining the times which produce the highest quality results in quadratic discriminant analysis (see in this description). The inventors have for example determined the percentage of correct classification using quadratic discriminant analysis, initially with a single parameter, as described in FIG. 15. Then the inventors worked with a combination of parameters, as described in FIG. 16. One can see that the observed variation in the percentages of correct classification allows those skilled in the art to easily verify whether the times t1 and t2 used are optimally chosen. Furthermore, those skilled in the art can easily determine a contingency table or a ROC curve for predetermined times t1 and t2, and thus associate a sensitivity/specificity with the method thus defined.

According to the invention, the method can be carried out on the basis of a blood or plasma sample previously obtained from a patient who may have or who does have a deficiency in at least one coagulation factor. According to some particular embodiments, the missing coagulation factor(s) is (are) chosen among: factor VIII, factor IX, factor XI, a bispecific antibody, or another hemophilia treatment, which can also be used as missing factor(s). According to other aspects, the method may be carried out on a sample:
i. From a patient being treated by supplementing with at least one coagulation factor, recombinant, modified, or possibly not, human or animal, chosen among: factor VIII (human or animal, modified, including recombinant, or possibly not), factor IX (in particular a concentrate of factor IX) (human or animal, modified, including recombinant, or possibly not), factor XI (human or animal, modified, including recombinant, or possibly not), factor XIII (human or animal, modified, including recombinant, or possibly not), and/or
ii. From a patient being treated with bispecific antibody treatment, for example emicizumab or Hemlibra® (Roche), or
iii. From a patient being treated with what is referred to as “bypassing” therapy, for example treated with factor VIIa, recombinant or non-recombinant, such as NovoSeven® (NovoNordisk), or
iv. From a patient being treated with therapy targeting both hemophilia A and hemophilia B, such as Fitusiran® (Sanofi) or Concizumab® (NovoNordisk) or Marstacimab® (Pfizer), or
v. From a patient being treated with antifibrinolytic therapy, for example tranexamic acid (Cheplapharm).

Fitusiran® is an RNAi complementary to antithrombin, a coagulation inhibitor.

Marstacimab® and Concizumab® are TFPI-specific antibodies, which also inhibit coagulation.

For a patient being treated with antifibrinolytic therapy, today this type of treatment is generally considered in case of dental extraction in severe, moderate, and minor hemophiliacs without inhibitors (HAS 2019, Steve Chaplin 2016 DOI 10.17225/jhp00085). This treatment is also known to prevent bleeding in persons with multiple traumas. The literature reports clinical use of tranexamic acid at doses ≥1 g/3× per day (Forbes et al. 1972 DOI 10.1136/bmj.2.5809.311) which can be considered equivalent to a dose ≥15 mg/kg in a hemophilia patient. Pharmacokinetic data for tranexamic acid show that at 15 mg/kg, the plasma concentration is 2 μg/mL (Steve Chaplin 2016 DOI 10.17225/jhp00085).

Although it is anticipated that the lysis curve of a fibrin clot remains usable at this plasma concentration of 15 mg/kg of tranexamic acid in a hemophiliac patient, according to one particular embodiment it is preferred that the plasma concentration of tranexamic acid in the tested sample does not exceed 5 μg/mL. Treatment with TXA leading to a plasma concentration of 5 μg/mL of tranexamic acid in the sample would lead to obtaining a DOD (delta optical density) at time t2 which is identical to the DOD (delta optical density) at time t1. Although a lysis curve can still be obtained, this situation would make it difficult to exploit the curve in order to reach a conclusion. However, monitoring remains possible, in particular for dosages giving low plasma concentrations of TXA, the method also allowing an appreciable variation in the plasma concentration of TXA, considering the dosages generally used.

According to one embodiment, the analyzed samples come from patients diagnosed with hemophilia. According to some particular embodiments, the patients are A-hemophiliacs (FVIII deficiency) or B-hemophiliacs (FIX deficiency) or C-hemophiliacs (FXI deficiency). The reference method for diagnosing hemophilia is to determine the level of Factor VIII, Factor IX, or Factor XI which allows confirming severe hemophilia when the level is below 1% of the normal expected level, moderate when it is between 1% and 5%, and minor when it is between 5% and 40% (levels of the missing factor presented above). However, as indicated above, the level of missing factor is not a predictor of bleeding risk. Two types of conventional methods exist: the “one stage clotting assay” method, which is derived from measuring the aPTT; or the “chromogenic assay” method, which is based on indirect measurement of FVIII by generating FXa and cleaving a specific substrate. One can refer to the literature: Srivastava et al. 2013 DOI 10.1111/hae.14046 and Srivastava et al. 2020 DOI 10.1111/hae.14046—§ Recommendation 3.2.9.

According to one particular embodiment, in the case where the analyzed sample comes from a patient who is being treated with an antifibrinolytic therapy which is tranexamic acid, and in particular, but not necessarily, this patient has been diagnosed with hemophilia, the sample analyzed in the method described here is a sample in which the plasma concentration of tranexamic acid in the tested sample does not exceed 5 μg/mL.

According to one embodiment, the blood or plasma sample is an undiluted sample of whole blood or plasma, in particular platelet-rich plasma or platelet-poor plasma, plasma containing microparticles of platelets, erythrocytes, or of any other cell, preferably a sample of platelet-poor plasma.

According to one particular embodiment, if the sample analyzed is a whole blood sample, t2 is chosen to be 650 seconds after t1.

According to one particular embodiment, if the analyzed sample is a plasma sample, t2 is chosen to be 600 seconds after t1.

According to one particular embodiment, in step a) of the method, the tissue factor, the phospholipids, the activator of the intrinsic coagulation pathway, tPA (tissue plasminogen activator), and optionally the coagulation factor that is missing in the analyzed patient, are mixed together beforehand then all of this is added to the blood or plasma sample to be analyzed, which is undiluted.

According to one particular embodiment, the sample is added to a volume of 200 μL, for a final volume of 300 μL of reaction mixture.

According to one particular embodiment, in step a., the tissue factor is present in the composition of reagents in an amount such that the final concentration of tissue factor in the mixture on which the kinetics measurement is carried out in step d. is between 0.01 and 5.0 pM.

According to one particular embodiment, in step a., the tissue factor is present in the composition of reagents in an amount such that the final concentration of tissue factor in the mixture on which the kinetics measurement is carried out in step d. is between 0.01 and 8.0 pM, in particular between 0.01 and 7.0 pM.

According to one particular embodiment where the analyzed sample is a sample of whole blood, in step a., the tissue factor is present in the composition of reagents in an amount such that the final concentration of tissue factor in the mixture on which the kinetics measurement is carried out in step d. is between 0.01 and 8.0 pM, or between 0.01 and 7.0 pM, or between 0.01 and 5.0 pM, or between 2.0 and 5.0 pM, and in particular is 2.0 pM.

According to one particular embodiment where the analyzed sample is a plasma sample, in step a., the tissue factor is present in the composition of reagents in an amount such that the final concentration of tissue factor in the mixture on which the kinetics measurement is carried out in step d. is between 0.01 and 1.0 pM, or between 0.1 and 0.7 pM, more preferably between 0.3 and 0.6 pM, and in particular is 0.5 pM.

According to one particular embodiment, in step a., the phospholipids are present in the composition of reagents in an amount such that the final concentration of phospholipids in the mixture on which the kinetics measurement is carried out in step d. is between 1 and 10 μM, or between 3 and 7 μM, more preferably between 3 and 5 μM, and in particular is 4 μM.

According to one particular embodiment, in step a., the activator of the intrinsic coagulation pathway, in particular one or more chosen among: ellagic acid, ethylene glycol gallate, silica, FIXa, FXIa, FXIIa, is present in the composition of reagents in an amount such that the final concentration of activator in the mixture on which the kinetics measurement is carried out in step d. is between 1 pM and 600 nM, or between 10 and 200 pM, more preferably between 75 and 200 pM, in particular is 100 pM, in particular the activator is FXIa at 100 pM.

According to one particular embodiment, in step a., the activator of the intrinsic coagulation pathway, when it is ellagic acid, ethylene glycol gallate, or silica, is present in the composition of reagents in an amount such that the final concentration of activator in the mixture on which the kinetics measurement is carried out in step d. is between 1 and 600 nM, or between 10 and 900 nM, or between 20 and 800 nM, or between 30 and 700 nM, or between 40 and 600 nM, or between 50 and 500 nM, or between 60 and 400 nM, or between 70 and 300 nM, or between 80 and 200 nM, or between 40 and 300 nM, in particular is 150 nM.

According to one particular embodiment, in step a., the activator of the intrinsic coagulation pathway, when it is FIXa, FXIa, or FXIIa, is present in the composition of reagents in an amount such that the final concentration of activator in the mixture on which the kinetics measurement is carried out in step d. is between 1 and 200 pM, or between 10 and 200 pM, more preferably between 75 and 200 pM, in particular is 100 pM; in particular the activator is FXIa at 100 pM.

According to one particular embodiment, in step a., the tPA (tissue plasminogen activator) is present in the composition of reagents in an amount such that the final concentration of tPA (tissue plasminogen activator) in the mixture on which the kinetics measurement is carried out in step d. is between 0.01 and 5 μg/mL, or between 0.1 and 2 μg/mL, in particular is 0.13 μg/mL.

According to one particular embodiment, in step a., the coagulation factor that is missing in the analyzed patient, in particular one or more chosen among: FVIII, FIX, FXI, is present in the mixture on which the kinetics measurement is carried out in step d., at a concentration between 0 and 2.0 IU/mL, or between 0 and 0.2 IU/mL, or between 0 and 0.1 IU/mL, or between 0 and 0.05 IU/mL, or between 0 and 0.025 IU/mL, in particular at a concentration of 0.015 IU/mL.

According to one particular embodiment, in step a., the coagulation factor that is missing in the analyzed patient may be another usual treatment for hemophilia, in particular chosen among one or more bispecific antibodies; for example emicizumab is present in the mixture on which the kinetics measurement is carried out in step d., at a concentration of between 0 and 80 μg/mL, or between 0 and 50 μg/mL, or between 0 and 20 μg/mL, in particularly at a concentration of 15 μg/mL.

It is understood that the missing factor may or may not be present. Indeed, although a “missing factor” is deficient in a patient, it will be understood that a patient diagnosed with hemophilia is generally treated to compensate for this absence, by administering the missing factor, so that it is expected that the analyzed sample may comprise a concentration as mentioned above without any particular procedure for this purpose. Conversely, it is also possible, according to one particular embodiment, to provide an amount of missing factor by extrinsic contribution to the mixture to be analyzed, or systematically to supplement the samples to be analyzed by extrinsic contribution to the mixture to be analyzed. Without being essential to the implementation of the method described herein, a homogeneous supplementation can in fact make it possible to normalize the results.

According to one particular embodiment, in step b. the incubation of the mixture obtained from step a. is carried out between 2° and 39° C., preferably at 37° C., for 2 to 10 minutes, in particular 5 minutes, in particular at 37° C. for 5 minutes, then calcium ions are added to the incubated mixture in an amount allowing a final concentration of calcium ions that is between 5 and 25 mM, preferably 17 mM.

According to one particular embodiment, the blood or plasma sample obtained from the patient and used for the method has a volume of between 5 μL and 500 μL, preferably between 50 UL and 400 UL, preferably between 50 μL and 300 μL, preferably between 100 UL and 300 μL, preferably approximately 200 μL.

According to some distinct particular embodiments, the degradation kinetics of the fibrin clot due to its lysis in step d. of the method is obtained by any method for measuring the degradation kinetics of said fibrin clot due to its lysis, in particular a method chosen among: a viscoelastic method, a rheometric method, an acoustic method, an optical method, a waveform analysis method, a fluorometric method, a magnetic resonance method, a turbidimetric method, in particular is performed by turbidimetry (absorbance and transmittance) or by a viscoelastic method (for example ROTEM or Quantra).

The evolution of a fibrin clot, as utilized in the present method, is independent of the type of measurement method that allows its evolution kinetics to be tracked. The evolution necessarily goes through a stage of degradation of the fibrin clot due to its lysis. The method described herein is based on utilizing the degradation kinetics of the fibrin clot during lysis. A lysis curve is generally monitored from the area corresponding to the end of formation of the fibrin clot, over time, until the return to the initial amplitude, meaning the amplitude observable before generation of the fibrin clot. It follows from the above that any method which allows monitoring the evolution of the fibrin clot lysis, in particular during a sufficiently long time to observe a return to the initial amplitude, can be suitable for implementing the invention as described here. The literature includes examples of implementing various methods in a conventional manner. Some examples are cited here. This patent application provides examples using a turbidimetric method which allows measuring the optical density (also called absorbance) of the analyzed sample over time. Also presented are results obtained by a viscoelastic measurement method (where a signal amplitude is measured in millimeters), in whole blood. However, these embodiments are not limiting, since the principle on which the invention is based, namely the observation of lysis kinetics by means of such a curve, and a subsequent treatment on the basis of values extracted therefrom, are independent of the measurement method employed. In an original manner, the measurement method of the invention takes into account as relevant parameters not only the degradation of a fibrin clot in an analyzed sample, but also the basal level of said clot. Unlike the parameters in the literature, the parameters defined for implementing the present invention, as carried out in measurement step e. described herein, surprisingly turn out to be the most differentiating for defining separate groups of samples, and therefore serve as classifiers for this purpose. The different groups obtained can then be used for other objectives.

As indicated above, observation of the clot lysis is carried out, in a conventional manner, over a time which includes the return to the initial amplitude, the clot then being completely degraded. The time to return to the initial amplitude can vary according to the measurement method used, and is therefore not limiting. The observation will be continued for as long as necessary.

According to one particular embodiment involving a method of measurement by turbidimetry, in particular by measurement of the Delta Optical Density (DOD) of the analyzed sample (also called Absorbance in the literature, with arbitrary values because they are dimensionless, linked to the type of device used and to a control sample for calibration), the measurement of the degradation kinetics is carried out at a wavelength between 350 and 800 nm, preferably at 540 nm, and for a duration of between 1400 and 3600 seconds, or between 1400 and 2400 seconds, starting from the triggering of coagulation by the addition of calcium ions. Note that in the present description, the expression “DOD” is used to mean this measured change in optical density, or “delta optical density”. This requires measuring optical density (OD) on a (lysis) curve in order to determine its value on the curve. The expressions “DOD parameter” or “DOD parameter at two times” are also employed and mean the same thing, i.e. the measurement of an Optical Density value, the invention being based on measuring two deltas in optical density values (DOD) at two different times on a lysis curve as measured in the invention. Measuring an optical density parameter at a given time therefore corresponds to measuring a “DOD” parameter in the present description.

According to one particular embodiment involving a measurement method by viscoelastometry, in particular by measuring a curve amplitude in millimeters, measurement of the degradation kinetics is carried out for a duration of between 1400 and 3600 seconds starting from the triggering of coagulation by the addition of calcium ions.

According to the invention, two values are determined in step e. of the method during this observation period: a basal level value of the fibrin clot at a time t1 of lysis and a degraded level value of the fibrin clot at a time t2, time t2 being subsequent to time t1.

The measurement made at time t1 corresponds to a value reflecting the end of polymerization of the clot (after its stabilization and before or at the start of its lysis), or the initial phase of lysis of the fibrin clot, t1 being chosen within the plateau portion or at the beginning of the lysis curve. Indeed, t1 is a time located between Tmax and TL of the lysis curve of the fibrin clot, which can therefore be located either in the plateau (between Tmax and T90% of the max) of the curve, or after the plateau (between T90% of max and TL).

The measurement made at time t2 corresponds to a value reflecting the degraded or partially degraded fibrin clot.

It is known from the prior art to measure, on lysis curves of a fibrin clot over time, mathematical parameters conventionally used in the field, which can be values measured at precisely defined times, or time ranges (time interval between two time limits). For example, we know the total lysis time TLT (time interval) which is the length of time between to (defined above) until the moment when the measured values return to their base level and no longer change, which corresponds to complete dissolution of the clot, or the fibrin lysis rate which is a slope, namely the slope of the descending curve of the lysis curve of the fibrin clot between the end of the plateau and the final time of the TLT, or the clot lysis time CLT (time interval) which has been defined as the length of time between the time corresponding to the moment when the ascending curve is at half of its value (before the peak and the plateau) and the time corresponding to the moment when the descending curve is also at half of its value (between the peak and the return to the base threshold).

However, none of these parameters defined in the literature have proven to be relevant as the most differentiating parameter for defining separate groups of samples, and therefore serving as classifiers for this purpose. Conversely, the present invention proposes, in an original manner, using as a new parameter for this purpose, the values obtained to quantify the basal level of the fibrin clot and the degraded level of the fibrin clot, respectively measured at two times, namely at t1 and t2 described herein. These two parameters result from an approach guided by patient samples (and not mathematically predefined), although due to the analysis carried out on a number of samples, they become independent of the particular characteristics of a given patient.

Refer to the above description concerning a more fine-tuned determination of times t1 and t2 that it is possible to retain. Those skilled in the art can easily verify whether the chosen times t1 and t2 are appropriate. t1 and t2 may also vary as a function of the concentration of the activator tPA (tissue plasminogen activator) used in vitro in the method, and possibly the type of method used (for example turbidimetric or viscoelastic method). Times t1 and t2, however, remain chosen within the measurement ranges described here, and can be optimized by verifying the values which give the best classification percentages in quadratic discriminant analysis, in particular under the following conditions for measuring the sample:
A) For a turbidimetric method: TF (tissue factor) at 0.5 pM and PPL (phospholipids) at 4 μM are mixed with FXIa at 100 pM, and tPA (tissue plasminogen activator) at 0.13 μg/mL (final values of the plasma test),
B) For a viscoelastic method: TF (tissue factor) at 2.2 pM and PPL (phospholipids) at 8 μM are mixed with FXIa at 200 pM, and tPA (tissue plasminogen activator) at 0.07 μg/mL (final values of the whole blood test).

According to one embodiment where the measurement is made by turbidimetry, time t1 is chosen to be within the interval between 700 and 900 seconds after coagulation is triggered by the addition of calcium ions, and time t2 is chosen to be within the interval between 1100 to 1400 seconds after coagulation is triggered by the addition of calcium ions.

According to one particular embodiment, which is preferred in the case of turbidimetry, t1 is defined to be between 700 and 800 seconds, preferably is defined to be at 750 seconds, and t2 is defined to be between 1300 and 1500 seconds, preferably is defined to be at 1400 seconds.

In one particular embodiment where the measurement is made by turbidimetry, t1 is defined to be at 750 seconds after coagulation is triggered by the addition of calcium ions and t2 is defined to be at 1400 seconds after coagulation is triggered by the addition of calcium ions.

Note that in a viscoelastic measurement method, the choice of times t1 and 2 may be made as in the turbidimetric method as described above, on the basis of the correct classification in quadratic discriminant analysis. Indeed, as indicated in FIG. 17, the number of correct classifications obtained is 100% by taking the same time values as those used in the turbidimetric method (750 sec and 1400 sec). Therefore the times indicated above for a method where the measurement is made by turbidimetry can also be transposed to a method using viscoelastic measurement.

According to one particular embodiment where the measurement is made by a viscoelastic measurement method, time t1 is chosen to be within the interval of 900 to 1200 seconds after coagulation is triggered by the addition of calcium ions, and time t2 is chosen to be within the interval of 1500 to 1800 seconds after coagulation is triggered by the addition of calcium ions.

According to one particular embodiment where the measurement is made by a viscoelastic measurement method, t1 is defined to be at 900 seconds after coagulation is triggered by the addition of calcium ions and t2 is defined to be at 1500 seconds after coagulation is triggered by the addition of calcium ions.

According to one particular embodiment where the measurement is made by a viscoelastic measurement method, t1 is defined to be at 1200 seconds after coagulation is triggered by the addition of calcium ions and t2 is defined to be at 1800 seconds after coagulation is triggered by the addition of calcium ions.

The values obtained (measured on the curve, or measured at t1 and t2) to quantify the basal level of the fibrin clot and the degraded level of the fibrin clot, constitute parameters of the degradation kinetics of the fibrin clot, measured at two times. The measurements carried out at t1 and t2 make it possible, regardless of how they are expressed (for example, absorbance for a turbidimetric method or amplitude in millimeters for a viscoelastic method), to provide information concerning both the degradation of the clot during lysis but also the basal level of the fibrin clot, measured at t1, which “reflects the end of polymerization of the clot” in the initial state. Indeed, as shown in FIG. 2, the lysis kinetics experimentally observed allow distinguishing between several different profiles.

The group numbered 3 (Group 3) in FIG. 2 and in the present description, has a low basal level for the fibrin clot and shortened lysis.

The group numbered 2 (Group 2) in FIG. 2 and in the present description, has an intermediate basal level for the fibrin clot and normal lysis.

The group numbered 1 (Group 1) in FIG. 2 and in the present description, has a high basal level for the fibrin clot and delayed lysis.

Thus, according to another aspect of the invention, a method is proposed for the in vitro measurement of fibrin clot degradation based on a curve of the lysis of a fibrin clot over time in a blood or plasma sample previously obtained from a patient who may have a deficiency in at least one coagulation factor, or any patient as defined in the present description, the method comprising the following steps:
a. Implementing a method for performing an in vitro measurement of a curve of the lysis of a fibrin clot over time according to any of the embodiments described herein, in particular involving a measurement by turbidimetry, on a blood or plasma sample obtained from said patient, at at least one given time and possibly at another or several subsequent time(s), and
b. Classifying the sample tested in step a. into a group reflecting a lysis profile of a fibrin clot (this step corresponding to an additional step f. of classifying the tested sample into a group reflecting a lysis profile of a fibrin clot), and
c. Optionally, on the basis of the classification obtained in step b., making a conclusion concerning the deficiency in coagulation factor(s) of the analyzed patient observed by the classification method or concerning the state of health of said patient, and possibly a conclusion concerning the evolution of said deficiency in coagulation factor(s) of the analyzed patient or of the state of health of said patient if several classifications carried out at separate and successive times are available.

Such a method is therefore, according to another formulation, a method for in vitro measurement of fibrin clot degradation and for classification of the blood or plasma sample which allowed establishing a curve of the lysis of a fibrin clot over time, comprising at least the following steps:
a. Implementing a method for performing an in vitro measurement of a curve of the lysis of a fibrin clot over time according to any of the embodiments described herein, in particular involving a measurement by turbidimetry, on a blood or plasma sample obtained from said patient, at at least one given time and possibly at another or several subsequent time(s), and
b. Classifying the sample tested in step a. into a group reflecting a lysis profile of a fibrin clot (this step corresponding to an additional step f. of classifying the tested sample into a group reflecting a lysis profile of a fibrin clot), and
c. Optionally, step c. described in the previous paragraph.

Such a method therefore yields a classification into a group, where appropriate a predetermined group (or “grouping” in equivalent terminology also used in this text), after the measurement of parameters which allow differentiating between several groups in a relevant manner, as provided by the present invention and presented above in an introductory manner.

Note that the classification into a group may be carried out according to a classification model that is predefined on the basis of classification parameters obtained with training data processed under the same experimental conditions as those of the analyzed sample. An example of such an embodiment is described in the experimental section. The quality of the classification made can be evaluated by verifying the number of correct classifications (as a percentage for example, as described herein).

According to another embodiment, the classification into a group may be carried out using decision thresholds, which can be associated with sensitivity and specificity measurements (which are associated with the classification made). As illustrated in the examples, establishing a ROC curve (conventional in the field) for a particular test (in particular a test with given predetermined times t1 and t2) makes it possible to define a threshold value allowing for example the inclusion of a sample in a group with a given sensitivity and specificity. An example of such an embodiment is also described in the experimental section. Once thresholds have been defined for a particular implementation of a measurement method according to the invention, such an embodiment can replace a classification into a group according to a predefined classification model as explained above. Although associated with a given sensitivity and specificity in relation to the decision made, such an embodiment is a practical alternative to implementing a predefined classification model on the basis of classification parameters obtained with training data processed under the same experimental conditions as those of the analyzed sample, in particular if the sensitivity and specificity are good.

In this regard, one will note that this patent application also envisages a method for the in vitro measurement of fibrin clot degradation on the basis of a curve of the lysis of a fibrin clot over time in a blood or plasma sample previously obtained from a patient who may have or who does have a deficiency in at least one coagulation factor, and for the classification of the blood or plasma sample which allowed establishing a curve of the lysis of a fibrin clot over time, the method comprising the following steps:
a. Mixing the sample previously obtained from said patient with a composition of reagents comprising tissue factor, phospholipids, tPA (tissue plasminogen activator), and one or more activator(s) of the intrinsic coagulation pathway, in particular one or more of these activator(s) chosen among: ellagic acid, ethylene glycol gallate, silica, FIXa, FXIa, FXIIa, and optionally one or more coagulation factor(s) that are missing in the analyzed patient, in particular chosen among: FVIII, FIX, FXI, a bispecific antibody, or another hemophilia treatment, which can also be used as missing factor(s), or a mixture of two or more of these factors and treatment(s);
b. Incubating the mixture obtained in a., then
c. Triggering coagulation by adding calcium ions to the mixture incubated in step b. in order to allow a fibrin clot to form in the mixture, then allowing lysis of the formed clot, and
d. Measuring the degradation kinetics of the fibrin clot formed and degraded during lysis after step c., and
e. Determining a basal level value of the fibrin clot at a time t1, time t1 being chosen to be located between Tmax and TL of the curve of the lysis of the fibrin clot, and/or determining a degraded level value of the fibrin clot at a time t2 that is subsequent to time t1, time t2 being chosen to be 300 to 900 seconds after t1, and
f. Depending on the basal level value of the fibrin clot at a time t1 compared to a predefined threshold value for said level (for example determined on the basis of a pool of samples), classifying the sample into a group reflecting a lysis profile of a fibrin clot, or, depending on the basal level value of the fibrin clot at a time t1 compared to a predefined threshold value for said level (for example determined on the basis of a pool of samples), classifying the sample into a group reflecting a lysis profile of a fibrin clot.

According to another aspect, and according to another particular embodiment, step b. defined above, of classifying the sample tested in step a. into a group reflecting a lysis profile of a fibrin clot (this step corresponding to an additional step f. of classifying the tested sample into a group reflecting a lysis profile of a fibrin clot) can be carried out:
Via a predefined classification model based on classification parameters obtained with training data processed under the same experimental conditions as those of the analyzed sample, the classification being made on the basis of the values measured at t1 and t2 in step e. described in this patent application for the measurement method, and/or
By comparing the value measured at t1 and/or t2 (one or the other as appropriate, or both values may be measured) respectively to one or more predetermined threshold(s), in order to decide on assigning the sample to a predetermined group, for example to include the sample in group 1 (in the example provided in this patent application, this group was determined to have a high basal level for the fibrin clot and delayed lysis), and/or to include the sample in group 3 (in the example provided in this patent application, this group was determined to have a low basal level for the fibrin clot and shortened lysis).

Thus, according to one particular embodiment, a method according to the invention comprises an additional step f. of classifying the tested sample into a group reflecting a lysis profile of a fibrin clot, said lysis profile being determined on the basis of the values measured at t1 and t2 in step e., the classification being made into a group reflecting a lysis profile of a fibrin clot, in particular into three distinct groups.

In one particular embodiment using thresholds (above paragraph) and a turbidimetric method, the predetermined groups for classifying the samples are three in number, namely:
a. group 1: patient samples demonstrating an elevated basal level (DOD t1) for the fibrin clot (above the predetermined threshold) and an elevated degraded level (DOD t2) for the fibrin clot (above the predetermined threshold);
b. group 3: patient samples demonstrating a low basal level (DOD t1) for the fibrin clot (below the predetermined threshold) and a low degraded level (DOD t2) for the fibrin clot (below the predetermined threshold);
c. group 2: patient samples demonstrating an intermediate basal level (DOD t1) for the fibrin clot (below the predetermined threshold) and an intermediate degraded level (DOD t2) for the fibrin clot (above the predetermined threshold).

The experimental section describes an example of a classification using predefined thresholds, to which reference is made. The thresholds can be adapted, in particular, according to the desired sensitivity and specificity. As an example, the method according to the invention can allow achieving a sensitivity of at least 80%, at a value chosen among 80%, 81%, 82%, 83%, 84%, 85%, 86%, 87%, 88%, 89%, 90%, 91%, 92%, 93%, 94%, 95%, 96%, 97%, 98%, 99% or 100%, and/or a specificity of at least 80%, at a value chosen among 80%, 81%, 82%, 83%, 84%, 85%, 86%, 87%, 88%, 89%, 90%, 91%, 92%, 93%, 94%, 95%, 96%, 97%, 98%, 99% or 100%, according to all possible endpoint combinations, as appropriate.

Thus, in one particular embodiment, where the DOD values are measured by turbidimetry and the DOD values are measured at time t1 defined at 750 seconds and at time t2 defined at 1400 seconds, in particular under experimental conditions as indicated in the experimental section of this patent application (in particular with values of TF (tissue factor) at 0.5 pM, PPL (phospholipids) at 4 μM which are mixed with FXIa at 100 pM, and tPA (tissue plasminogen activator) at 0.13 μg/mL (final values of the plasma test) in step a. of the measurement method), then:
The predetermined threshold for the DOD measured at t1 is 1.14, and
The predetermined threshold for the DOD measured at t2 is 0.26.
   It follows that in this context, the groups are established as follows:
a. group 1: patient samples demonstrating a basal level (DOD t1) for the fibrin clot that is greater than or equal to 1.14 and a degraded level (DOD t2) for the fibrin clot that is greater than or equal to 0.26;
b. group 3: patient samples demonstrating a basal level (DOD t1) for the fibrin clot that is less than or equal to 1.14 and a degraded level (DOD t2) for the fibrin clot that is less than or equal to 0.26;
c. group 2: patient samples demonstrating a basal level (DOD t1) for the fibrin clot that is less than 1.14 and a degraded level (DOD t2) for the fibrin clot that is greater than 0.26.

A person skilled in the art, by comparing with the guidance elements provided above, will be able to transpose this scale to other measured values according to the context.

According to one aspect, a measurement method as described herein is disclosed, comprising an additional step f. of classifying the tested sample into a group reflecting a lysis profile of a fibrin clot, said lysis profile being determined on the basis of the values measured at t1 and t2 in step e. described herein for the measurement method, in accordance with a classification model predefined on the basis of classification parameters obtained with training data processed under the same experimental conditions as those of the analyzed sample. In one particular embodiment, the classification of the tested sample into a group reflecting a lysis profile of a fibrin clot is carried out on the basis of a classification model obtained by unsupervised or supervised learning. In an even more particular embodiment, the classification model is obtained by a learning method chosen among: a hierarchical analysis, in particular a centered and normalized hierarchical analysis, a K-means method, a quadratic discriminant analysis, logistic regression, or a random forest method.

Indeed, according to the experimental section and in parallel with observing the aforementioned differences in lysis profile, the inventors were able to reveal, statistically, three groups of samples among those tested, on the basis of the two DOD parameters at two times as reported herein, using a centered and normalized hierarchical analysis, as is also indicated in the experimental section. Finally, a classification into three patient groups was confirmed on another set of data from new patients selected at another site.

According to one embodiment, a method according to the invention allows classifying samples into three (3) distinct groupings, as defined above in the description, either by using a classification model or by using thresholds, or both.

However, it is anticipated that this number of three groups retained in this patent application concerning the sets of analyzed samples for which the results are presented, may change depending on the samples retained. It is indeed conceivable, for example when samples with a 0% level of missing factor are included, to have more groups in order to allow differentiating the untreated plasmas in particular.

For example, with the database used for the results described herein, and a criterion of 90% similarities used in a normalized centered hierarchical analysis, a total of 8 groups could be highlighted. Conversely, implementing a complete normalized hierarchical analysis in 2 groups made it possible to separate out group no. 3 (with the 0% concentrations of group no. 2) from the other two groups.

The inventors then obtained a correct classification rate of 100% in quadratic discriminant analysis, and 99% (3 errors) by the normalized K-means method (analyses carried out in Minitab).

This is how, in addition to the fact that they allow characterizing a behavior (profile) of a fibrin clot in a patient, the data pairs composed of the values obtained at times t1 and t2, and the times t1 and t2, can also, and according to another aspect of the invention, serve as classifiers for the purpose of distinguishing between the different profiles mentioned above. The parameters determined in the present invention can therefore make it possible, according to another aspect of the invention, to classify an analyzed sample according to the behavior of the clot during lysis, and in particular according to the degradation during lysis, but also according to the basal level of the fibrin clot, measured at t1, which “reflects the end of polymerization of the clot”, compared to a training group constructed under the same conditions.

In this text, the terms “classification” and “classifying” can be used interchangeably and are synonymous.

According to the invention, any unsupervised method of analysis can be used for the classification into groups. Such a method may, if necessary, be supplemented by any supervised method for verifying the classification. Similarly, any supervised method of analysis can be used to predict a classification.

In this regard, when an unsupervised statistical learning method is used, the number of groups is an input data for carrying out the method, which allows those skilled in the art to adjust the implementation of the method described herein according to the needs of the investigation. The elements presented here allow those skilled in the art to choose the most appropriate embodiment for the needs of their investigation.

According to different independent embodiments, a classification model can be used, where appropriate, to define groups.

A classification model is obtained by an unsupervised or supervised learning method trained on training data processed under the same experimental conditions as those which are applied to the blood or plasma sample to be analyzed by the method described herein.

According to the invention, an unsupervised method of analysis can be chosen among: a hierarchical analysis, in particular a centered and normalized hierarchical analysis, or a K-means method.

The relevant parameters for the implementation of such unsupervised methods are:
a. The number of groups or similarities
b. The method of connection
c. The distance measurement
d. The normalization

In the experimental section of this patent application, the parameters which were used are: a: 3 groups, b: centered connection, c: Euclidean distance, and d: normalized values. Those skilled in the art can refer to the literature, or even simply to the documentation of the Minitab software (Observations in Groups—General Information) for the implementation of such a method with its various parameters.

According to the invention, a method of supervised analysis may be chosen among: a (quadratic) discriminant analysis, logistic regression, or a random forest method.

The relevant parameters for the implementation of such supervised methods are:
a. The discriminant function: Linear or quadratic
b. Random forest method: the number of factors per tree/number of trees
c. Logistic regression: multinomial (logistic regression)/reference group

In the experimental section of this patent application, the parameters which were used are: a: quadratic discriminant analysis (bibliographic reference: Minitab software documentation (General Information)), b: two factors per tree, 3000 trees (bibliographic reference: Leo Breiman 2001 Random Forest, Machine Learning 45, 5-32), c: Multinomial logistic regression with group no. 2 selected as the reference group (similar analysis obtained with reference group no. 1 or no. 3) (bibliographic reference: R software: Fit Multinomial Log-linear Models (r-project.org) and Venables and Ripley 2002 Modern Applied Statistics with S. Fourth edition).

Those skilled in the art can refer to the above literature for the implementation of these methods, with their different parameters.

The groups obtained by learning reflect different degradation behaviors due to fibrin clot lysis in patients as represented in the training database. It is therefore possible for a practitioner, in an additional step once a classification has been made, to make a conclusion concerning the deficiency in coagulation factor(s) of the analyzed patient observed by the classification method on the basis of the classification which has been made, or a conclusion concerning the state of health of said patient on the basis of the classification which has been made, and possibly a conclusion concerning the evolution of said deficiency in coagulation factor(s) of the analyzed patient or the state of health of said patient if several classifications made at separate and successive times are available (these demonstrating an evolution).

According to one particular embodiment, the method of supervised learning used is a method of discriminant analysis, more particularly, a method of quadratic discriminant analysis. The characteristics of implementing such a method are known in the literature. It can be implemented by many statistical tools or software, for example Minitab 18 and R.

Statistical analysis of all the samples is carried out using statistical software, preferably Minitab 18 and R software.

The training data may be defined in a conventional manner by those skilled in the art. In the current case, the experimental section provides complete guidelines for the sampling used to construct the test database.

The method of unsupervised or supervised statistical learning used results in the determination of a model or of classification parameters which are thus defined and therefore predetermined. In particular, the two parameters at t1 and t2 are used for the statistical analysis. These models or predefined classification parameters then make it possible, where appropriate, when carrying out the classification step of a method as described herein, to classify the analyzed sample into a group, according to the values measured for the analyzed sample at t1 and t2 in step e. (which serve as classifiers).

One can see in the experimental section that it was possible to reliably allocate each analyzed sample, in a context of prediction on the basis of a model, to a group reflecting a particular behavior or lysis profile of the fibrin clot. In particular, FIG. 4 shows that it was possible to define three groups without any overlap between them.

The three groups denoted 1 to 3 which were defined in the experimental section, by means of a method of turbidimetric measurement under the conditions mentioned above for defining times t1 and t2, for plasmas from hemophilia-A patients, correspond to three typical profiles of fibrin degradation in this group of patients, as shown in FIG. 2. They each have their own fibrinogen level (FIG. 5 and FIG. 9). Group 3 defined here is the group which has a shortened lysis time. It is then possible for a practitioner, in an additional step once a classification has been carried out, to make a conclusion concerning the deficiency in coagulation factor(s) of the analyzed patient observed by the classification method, on the basis of the classification which has been made, or a conclusion concerning the state of health of said patient on the basis of the classification which has been made, and possibly a conclusion concerning the evolution of said deficiency in coagulation factor(s) of the analyzed patient or of the state of health of said patient if several classifications carried out at separate and successive times are available (these demonstrating an evolution).

According to another aspect, it is also conceivable to provide a method for determining a clinical phenotype of a patient, based on the patient's fibrin degradation profile, in particular to have a bleeding clinical phenotype which indicates the bleeding tendency of said patient. Indeed, it is anticipated that a patient's predisposition to bleeding differs in line with the various profiles analyzed here. According to this aspect, determining the clinical phenotype, in particular the bleeding clinical phenotype, takes advantage of the implementation of a method such as described herein involving at least one step e. as described herein for determining values at times t1 and t2, as set forth in this patent application.

According to one aspect, the determination of the clinical phenotype, in particular the bleeding clinical phenotype, also takes advantage of a classification made as described herein and set forth in this patent application.

In fact, depending on the classification that can be made on the basis of their group reflecting the degradation profile of their fibrin clots after the lysis of said clots, it is anticipated that the plasmas of the analyzed patients will react differently to the therapeutic treatment which the patient will undergo (supplementing with the missing factor or administering an antifibrinolytic). Reference is made in particular to the experimental data corresponding to FIGS. 7A and 7B.

One can also see, according to the experimental results obtained, that after their classification in Group 2, two patients experiencing bleeding changed treatment and went from on-demand treatment to preventive treatment. This change in treatment protocol allowed reducing the ABR in both cases.

Thus, according to another aspect of the invention, a method is proposed for monitoring a therapeutic treatment administered to a patient who may have or who does have a deficiency in at least one coagulation factor, comprising the following steps:
a. implementing a method for performing an in vitro measurement of a curve of the lysis of a fibrin clot over time according to any of the embodiments described herein, in particular involving a measurement by turbidimetry, on a blood or plasma sample obtained from said patient, at at least one given time and possibly at another or several subsequent time(s), said method including a step f. of classifying the tested sample into a group reflecting a lysis profile of a fibrin clot; and
b. on the basis of the classification obtained for the analyzed sample into a group reflecting a lysis profile of a fibrin clot in said patient, making a conclusion concerning the deficiency in coagulation factor(s) of the analyzed patient observed by the classification method or concerning the state of health of said patient, and possibly a conclusion concerning the evolution of said deficiency in coagulation factor(s) of the analyzed patient or of the state of health of said patient if several classifications carried out at separate and successive times are available.

By “patient who may have or who does have a deficiency in at least one coagulation factor” or “blood or plasma sample obtained from said patient” or “classification obtained for the analyzed sample into a group reflecting a lysis profile of a fibrin clot in said patient”, these are in reference to the definitions and descriptions provided above in the context of the method described above. The related descriptions also apply to this aspect, and the features set forth throughout this description may be incorporated independently of each other, or in combination with each other, into the present aspect.

Hemophilia patients are generally treated for their condition and therefore follow a maintenance therapy for which the dosage is not necessarily always optimized, in particular for the purposes of bringing them to a clinical profile of reduced risk such as, for example, annual bleeding rate, or simply to ensure an improvement in their quality of life. This is where the classification made possible by the present invention allows personalizing the trough level for the maintenance therapy, or considering additional treatment with antifibrinolytics, or adjusting the treatment(s) for these purposes, as indicated in the experimental section for hemophilia-A patients.

The types of possible primary therapies for patients with hemophilia A, B, or C mainly consist of the administration of factor VIII, IX, or XI, respectively. Known commercial formulations of factor VIII include in particular the drugs Advate® (Shire), Elocta® (Sanofi), and Jivi® (Bayer).

Also known for hemophilia-A patients are treatments with bispecific antibodies, in particular the drug Hemlibra® or Emicizumab® (Roche).

Antifibrinolytic treatments are also known, in particular the administration of tranexamic acid (TXA), a synthetic derivative of lysine (the drugs Lysteda® or Cyklokapron® (United States), Transamin® or Transcam® in Asia, Espercil® in South America, and Exacyl® or Spotof® in Europe), prescribed in cases of excessive bleeding and intended to inhibit the fibrinolysis system. Tranexamic acid blocks the binding of the lysine residue of fibrin by plasmin, the enzyme responsible for fibrinolysis. The normal action of plasmin, namely dissolving the clot (fibrinolysis), is thus blocked. It is prescribed in moderate to minor hemophilia without inhibitors or in the event of surgery (HAS 2019).

We will see in the experimental section (FIG. 7) that providing TXA in addition to a maintenance therapy makes it possible to modify the lysis kinetics of a fibrin clot. In particular, the lysis kinetics after adding TXA to the maintenance therapy becomes slower. Therefore, in this particular case it is group 3 of the patients described here, with a shortened lysis time, who would benefit most from adjusting their treatment by adding TXA. Indeed, it becomes possible to move these patients into group 2 after a treatment adjustment (FIG. 7). Similarly, the invention makes it possible to consider personalizing the trough level for the maintenance therapy, according to the initial classification or at a given time in a treatment protocol.

“Conclusion concerning the state of health of said patient” thus refers to the conclusions made possible based on classification into a group reflecting the lysis profile of a fibrin clot in a patient, in particular concerning the question of whether the clot lysis behavior is abnormal.

Alternatively, the method described here simply makes it possible to qualify the lysis profile of a fibrin clot in a patient as a physiological parameter, by classification in a group, making it possible where appropriate to propose adjustments to dosages or treatments in order to vary this physiological parameter, particularly in relation to pre-established criteria.

“Conclusion concerning the evolution of the state of health of said patient if several classifications carried out at separate times are available” refers to reiterating the classification at one or more time intervals in the same patient, in order to evaluate, by comparison with the previous data available, the evolution of the situation of said patient, with regard to modifying or not modifying his treatment, as appropriate. For example, a patient may or may not change group after a treatment. Similarly, a patient may or may not change group after an absence of treatment.

According to one particular embodiment, the monitoring method further comprises a step of adjusting the therapeutic treatment followed by said patient, according to the classification obtained. Results demonstrating the relevance of the approach are described in the experimental section.

For example, a change of treatment (adjustment to the therapeutic treatment followed by the patient) for two patients classified in Group 2, from treatment on demand to preventive treatment, in both cases made possible a reduction in their ABR and therefore in their bleeding.

For example, the administration of an antifibrinolytic treatment can be offered to a patient whose sample has been classified in group 3, with the aim of prolonging the lysis time of the fibrin clot in this patient. In the case of patients in group 1 or 2, an adjustment of the maintenance therapy (dosage for the administration of the missing factor) may be proposed, in order to optimize the treatment with regard to the effects obtained on the lysis of fibrin clots. According to another example, both an antifibrinolytic treatment and an adjustment of the maintenance therapy may be offered.

According to some particular embodiments, the adjustment of the therapeutic treatment followed by said patient, according to the obtained classification, may consist of:
a. If the classification turns out to be associated with a high risk of bleeding: evaluating the advisability of or transitioning to a maintenance therapy supplemented by an antifibrinolytic treatment, for example tranexamic acid (TXA);
b. If the classification turns out to be associated with a low risk of bleeding: evaluating the advisability of, or adjusting the maintenance therapy by, reducing the dosage;
c. If the classification turns out to be associated with a moderate risk of bleeding: evaluating the advisability of, or adjusting the maintenance therapy by, increasing the dosage.

One particular example which offers proposals for specific adjustments is included in the experimental section (Table 3): the proposed treatments can be combined with the general presentation made above.

According to another aspect, it is proposed to employ an antifibrinolytic agent, in particular tranexamic acid (TXA) or a composition comprising an antifibrinolytic agent or tranexamic acid (TXA), for use in treating a patient who may have or who does have a deficiency in at least one coagulation factor, in particular a patient diagnosed with hemophilia, said use comprising the carrying out of a measurement method on a sample from said patient, according to one any of the embodiments described herein, where appropriate with classification of the sample, or monitoring or adjustment of the therapeutic treatment of said patient.

The above definitions apply identically in the context of this aspect.

Examples of antifibrinolytic agents are given in this patent application: any agent indicated as such falls within the scope of this disclosure.

According to one particular embodiment, the use of an antifibrinolytic agent, in particular tranexamic acid (TXA) or a composition comprising an antifibrinolytic agent or tranexamic acid (TXA), in the treatment of a patient for whom a classification or monitoring method according to any of the embodiments described herein has been carried out on their blood or plasma sample, applies to the case where the tested blood or plasma sample has been classified into a group indicating a reduced lysis time of a fibrin clot, of the group 3 type described herein (determination of a high risk of bleeding-see experimental section, combinable with the general presentation made here).

The invention also relates to a method for screening a therapeutic molecule or a treatment for hemophilia, in order to determine whether said therapeutic molecule or said treatment allows modifying the lysis profile of a fibrin clot, comprising the following steps:
a. Implementing a method for performing in vitro a measurement of a curve of the lysis of a fibrin clot over time according to any of the embodiments described herein, on a blood or plasma sample previously obtained from said patient at a given time, and optionally classifying the blood or plasma sample into a group reflecting a lysis profile of a fibrin clot, and
b. Implementing a method for performing an in vitro measurement of a curve of the lysis of a fibrin clot over time according to any of the embodiments described herein, on a blood or plasma sample previously obtained from said patient at a given time that is different from the time in step a., after administering to said patient a therapeutic molecule or treatment aimed at modifying his or her lysis profile of a fibrin clot and where appropriate also treating a hemophilia condition, and optionally classifying the blood or plasma sample into a group reflecting a lysis profile of a fibrin clot, and
c. Comparing the curves of the lysis of a fibrin clot over time which are obtained in steps a. and b., and optionally comparing the classifications obtained in steps a. and b.,
d. Optionally, making a conclusion concerning the ability of the therapeutic molecule or treatment to modify the lysis profile of a fibrin clot, after comparing the curves of the lysis of a fibrin clot over time obtained in steps a. and b, and optionally after comparing the classifications obtained in steps a. and b.,
e. Optionally, repeating the above steps after modifying the treatment administered to the patient.

Such a method can be carried out in vitro for steps a. and b., on samples previously obtained from patients.

The therapeutic molecules targeted may be therapeutic molecules for which illustrative examples are cited in this patent application, without limitation, or any treatment aimed at restoring, in particular endogenously, proper hemostasis in patients (e.g. corresponding to treatment for hemophilia). In particular, gene therapy based on repairing the faulty FVIII or FIX gene is one form of treatment. The modification introduced in step b. aimed at modifying the lysis profile of a fibrin clot in the patient, and where appropriate also treating a hemophilia condition, may concern the molecule administered, the dosage, the type of treatment (gene therapy if applicable), the dosage, or any combination of such parameters. The conclusion concerning the ability of the therapeutic molecule or treatment to modify the lysis profile of a fibrin clot can be assessed for example in the case where a change of group is observed (or not observed) for the sample but also by visually observing the lysis curves obtained in the two steps a. and b. The possible conclusions depend on the specific case, and guiding elements are provided by way of example in this description: those skilled in the art will be able to transpose these lessons to the reaching of a conclusion.

By extension, depending on the ability of the therapeutic molecule or of a treatment for hemophilia that is administered to the patient to modify the lysis profile of a fibrin clot in a favorable sense for the patient, which can be deduced from step d. above, the screening method could be considered a method of determining the treatment sensitivity of a patient for a hemophilia treatment, where appropriate modified as it was administered in step b., in comparison to a possible treatment administered in step a. According to one particular example, a patient may be determined as sensitive, in a favorable sense, to a treatment administered in step b., in comparison to a possible initial treatment or an absence of initial treatment, under the treatment conditions of step a., if the treatment in step b. allows a sample taken from said patient to move from group 1 or group 3 to group 2, in the case of classification into three groups.

Such a method for determining the sensitivity of a patient to a hemophilia treatment remains carried out in vitro for steps a. and b., on samples previously obtained from patients, and includes the following steps:
Carrying out steps a. to c. of a screening method as defined above, and
Concluding that the patient is favorably responsive to the treatment administered in step b., if the response obtained in step b. concerning the change to the lysis profile of a fibrin clot is considered favorable for said patient, or concluding that the patient is not favorably sensitive to the treatment administered in step b., if the response obtained in step b. concerning the change to the lysis profile of a fibrin clot is considered unfavorable for said patient, or even concluding a lack of sensitivity to the treatment if no sensitivity can be determined.

Furthermore, one will note that the step involving the lysis kinetics of a fibrin clot in the methods described herein may advantageously be carried out on a diagnostic machine, preferably a coagulation analyzer. Such a device may also encompass computer means for processing data, thus making it possible also to carry out with a single device or via appropriate means, possibly remotely, at least one other step of the methods described herein which can be implemented through a computer program or processor.

According to another aspect, there is thus proposed a data processing system or a device comprising means for implementing at least step e. of the method described herein or step f. of the classification method described herein, according to any one of the embodiments described, and where appropriate also at least one other of the steps described in an embodiment of this description, and optionally comprising means for providing as input and/or output the variables generated during these steps, in order to return the classification result which takes into account the variables provided as input, and optionally also comprising a device for measuring the degradation kinetics of a fibrin clot, or calling upon a device for measuring the degradation kinetics of a fibrin clot, in particular remotely, and optionally also comprising a processor adapted to implement said steps.

According to another aspect, a computer program is proposed, comprising program code instructions for executing the steps of a method according to any of the embodiments described herein when said program is executed on a computer. According to one particular embodiment, the computer program allows controlling the implementation of the steps of the method and/or carrying out, at least in part, the calculation operations for a classification or a conclusion relating to the monitoring.

According to one embodiment, the computer program comprises instructions which, when the program is executed by a computer, lead it to implement at least step e. of the method described herein or step f. of the classification method described herein, according to any embodiment, and where appropriate at least one other of the steps of the method described herein, and optionally instructions also allowing the variables generated during these steps to be retrieved as input and/or be provided as output.

According to one embodiment, the computer program comprises instructions which lead a data processing system or a device as described herein, in particular a device including a kinetics measurement device as defined in any of the embodiments described herein or a data processing system or a device calling upon such a measurement device, in particular remotely, to execute at least step e. of the method described herein or step f. of the classification method described herein, and where appropriate also at least one other of the steps of the method described herein, according to any embodiment, in particular step d. of the method described herein, according to any embodiment.

According to another aspect, a computer-readable storage medium is proposed, comprising instructions which, when executed by a computer, lead it to implement at least step e. of the method described herein or step f. of the classification method described herein, and where appropriate also at least one other of the steps of the method described herein, according to any embodiment, and optionally comprising means for providing as input and/or output the variables generated during these steps, and/or to return the classification result which takes into account the variables provided as input, and optionally also comprising means for giving instructions to a kinetics measurement device, in particular remotely, for the implementation of a method described herein, according to any embodiment.

According to another aspect, a computer-readable data medium on which the computer program described herein is stored, or a signal from a data medium carrying the computer program described herein, is proposed.

According to another aspect, a computer-readable non-transitory storage medium is proposed on which is stored a program for implementing the method described herein, according to any embodiment, when this program is executed by a processor.

According to another aspect, a kit is proposed, in particular adapted for implementing a measurement, classification, or monitoring method described herein, according to any embodiment, comprising:
a. one or more of the following reagents: tissue factor, phospholipids, an activator of the intrinsic coagulation pathway chosen among: ellagic acid, ethylene glycol gallate, silica, FIXa, FXIa, FXIIa, or several of these, tPA (tissue plasminogen activator), calcium ions,
b. Optionally, a missing coagulation factor chosen among factor VIII, factor IX, factor XI, or several of these, a bispecific antibody or another hemophilia treatment which can also be used as missing factor(s).
c. Optionally, bispecific antibodies, for example emicizumab or Hemlibra® (Roche), or several of these,
d. Optionally, one or more appropriate buffers,
e. Optionally, instructions for carrying out one or several degradation kinetics of a fibrin clot, and
f. Optionally, a system and/or device and/or computer program and/or computer-readable data medium according to any of the embodiments described herein,
g. Optionally, instructions which allow implementing the measurement, classification, or monitoring method according to any of the embodiments described herein,
h. Optionally, instructions relating to the use of a signal from a data medium, for the implementation of a measurement, classification, or monitoring method according to any of the embodiments described herein.

As part of such a kit, all the features disclosed in other parts of this description may be integrated, alone or in combination.

### BRIEF DESCRIPTION OF THE FIGURES

Other features, details, and advantages will become apparent upon reading the detailed description below, and upon analyzing the attached drawings, in which:

FIG. 1: shows an explanatory diagram of the Coagulation-Lysis test method according to the invention, commented on in the Examples: 1. 200 μL pure plasma; 2. 50 μL reagent TF tPA FXIa; 3. Incubation at 37° C.; 4. 50 μL calcium reagent, TO of the test; 5. OD measurements. 6. Post-processing software and statistical analysis

FIG. 2: shows typical fibrin degradation profiles of three different hemophilia-A plasmas.

FIG. 3: shows a chart of the sample analysis for the study as reported in the examples. Legend: 1. 26 spans of 10 levels of FVIII [0-100%]. 2. Unsupervised and supervised analysis. 3. Patient samples. 4. Supervised analysis.

FIG. 4: shows classification of patients according to the DOD parameter (delta optical density) at two times (Results by group).

FIG. 5: shows the fibrinogen level according to each of the groups.

FIG. 6: shows the differences in turbidity at two times, in the presence of plasmas with severe hemophilia A.

FIGS. 7A and 7B: show the combined effect of 1 μg/mL TXA in association with the missing factor for hemophilia A plasma with shortened lysis.

FIG. 8: shows the differences in turbidity at two times (phenotype study).

FIG. 9: shows the fibrinogen level by group (phenotype study).

FIG. 10: shows the fibrinogen level versus the DOD value (delta optical density) at time 1 (phenotype study).

FIG. 11: shows the lysis time by group (phenotype study).

FIG. 12: shows the lysis rate by group (phenotype study).

FIGS. 13A, 13B, and 13C: show a test on the ROTEM machine (viscoelastic method) at a concentration of 20% FVIII (0.2 IU/mL) (Groups 3, 2, and 1, respectively).

FIG. 14A: shows the differences in amplitude at two times, with ROTEM parameters A15 and A25.

FIG. 14B: shows the differences in amplitude at two times: at 20 minutes and 30 minutes (viscoelastic method).

FIG. 15: shows the correct classification percentages in quadratic discriminant analysis with a single parameter (Optical Density measured one time with a turbidimetric measurement method).

FIG. 16: shows the correct classification percentages in quadratic discriminant analysis with two parameters (Optical Densities measured two times with a turbidimetric measurement method), compared to a single parameter.

FIG. 17: shows the correct classification percentages in quadratic discriminant analysis with a single parameter (Optical Density measured one time with a viscoelastic measurement method).

FIG. 18: ROC curve for parameter DOD t1 in the inclusion of patients in group 1 (Curve obtained using the “Analyze-it” add-in).

FIG. 19: Sensitivity and specificity for group 1 according to the decision threshold in DOD t1 (Curve obtained using the “Analyze-it” add-in).

FIG. 20: ROC curve for parameter DOD t2 in the inclusion of patients in group 3 (Curve obtained using the “Analyze-it” add-in).

FIG. 21: Sensitivity and specificity for group 3 according to the decision threshold in DOD t2 (Curve obtained using the “Analyze-it” add-in).

FIG. 22: Computer simulation of +3% errors in DOD t1 and DOD t2 for 15 hemophilia-A patients

FIG. 23: Kinetics of DOD over time during clot lysis in plasmas from three hemophilia-A patients (HA1 6496, HA2 62025845, HA3 6352-donor identical to 3944) spiked with 2% factor VIII obtained with STA-Cephascreen activator (ethylene glycol gallate at 48 nM) or STA-PTT Automate (silica at 290 nM) and TF at 0.5 pM instead of FXIa.

FIG. 24: Evolution of DOD t1 and DOD t2 parameters for the three groups of hemophilia-A plasmas (HA1 6628, HA2 62025845, HA3 6352-donor identical to 3944) overloaded with 0, 3, 15, or 30 μg/mL emicizumab versus 0, 2, 20 or 100% FVIII (Advate®) as a reference.

FIG. 25: Boxplots of FXIII: Ag levels (%) according to the three groups of hemophilia-A and B patients

FIG. 26: Evolution of parameters DOD t1 and DOD t2 of three hemophilia-A plasmas overloaded at two concentrations of NovoThirteen® (+30 and +80%) and at 0, 2, and 20% FVIII (Advate®).

FIG. 27: Evolution of parameters DOD t1 and DOD t2 of two hemophilia-A plasmas overloaded with three concentrations of Susoctogog alfa (0, 2 or 10, 20 and 100%)

FIG. 28: Evolution of parameters DOD t1 and DOD t2 of three plasmas from hemophilia-B patients (B) overloaded with three FIX molecules.

FIG. 29: Evolution of parameters DOD t1 and DOD t2 of twelve plasmas from hemophilia-A patients (A) and five hemophilia-B (B).

FIG. 30: Evolution of parameters DOD t1 and DOD t2 of eight plasmas from hemophilia-A patients (A) with and without emicizumab 20 μg/mL and two hemophilia-B patients (B) with and without Rixubis® at 40%.

### EXAMPLES

### A. Coagulation-Lysis Test

Dynamic measurement of the formation and degradation of the fibrin clot is carried out on a set of samples. The test, called the “Coagulation-Lysis” test, is a turbidimetric, automated, encompassing test for measuring the formation and lysis of the fibrin clot. For the analysis, the invention takes advantage of only the “lysis” part of the fibrin clot formation and lysis curve. The test is performed by mixing the plasma sample, undiluted, with an intermediate reagent containing a very low concentration of tissue factor (TF), phospholipids (PPL), plasminogen activator (tPA), and activator of the intrinsic pathway, preferably factor XIa (FXIa). After incubating the mixture, the calcium reagent triggers the generation of thrombin and the formation of the fibrin clot which quickly begins to be degraded by the tPA (tissue plasminogen activator).

The test can be carried out by any existing instrument and in particular by a turbidimeter or spectrophotometer. According to one advantageous embodiment, the steps of the method are implemented on an STA-R Max or STA-R analyzer. According to one embodiment, the blood or plasma sample is preferably a platelet-poor plasma sample. It is obtained in particular by centrifuging a citrated tube comprising the patient's blood sample, for 15 minutes at a speed of 2000 to 2500 g, at a temperature between 18 and 22° C. The sample may also undergo a second centrifugation for 15 minutes, at a speed of 2000 to 2500 g, at a temperature between 18 and 22° C. This treatment is conventional.

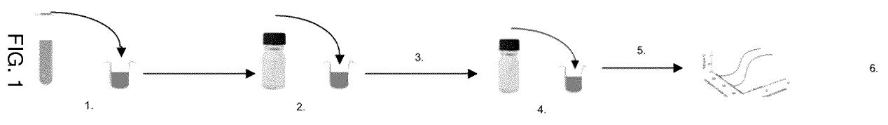

As indicated in FIG. 1, TF (tissue factor) and PPL (phospholipids) are mixed with an activator of the intrinsic pathway, preferably FXIa, and tPA (tissue plasminogen activator). According to one embodiment, TF (tissue factor), PPL (phospholipids), FXIa, and tPA (tissue plasminogen activator) are mixed at the following concentrations: 0.5 pM, 4 μM, 100 PM and 0.13 μg/mL final test.

An amount of the missing factor may be added to provide a final test concentration of between 1.5 and 200% (equivalent to 0.015 and 2.0 IU/mL). For this purpose, the missing factor may be added by the intermediate reagent according to the initial amount of factor in the sample (determinable).

The undiluted plasma sample, preferably 200 μL, is mixed with the intermediate reagent, preferably 50 μL.

Next is a step of incubating the mixture at 37° C. then adding the calcium ion-based triggering reagent to the mixture (FIG. 1), preferably 50 μL in 102 mM final reagent (17 mM final test), to initiate the thrombin generation and fibrin clot formation (FIG. 1).

Then, the change in optical density is read (FIG. 1) dynamically (every 2 seconds) at a single wavelength between 350 and 800 nm, preferably at 540 nm and preferably for a duration of 1986 seconds (33 minutes).

The “fibrinogram” is the curve which shows the evolution of the physical properties of the clot, preferably the evolution of the optical properties of the clot, over the course of the fibrin formation and until its lysis. Only the lysis of the clot is required for analyzing a profile in the context of the present invention.

Lysis is monitored by the DOD (delta optical density) over time until the return to the initial amplitude. The kinetics in the DOD (delta optical density) over time thus obtained make it possible to calculate DOD parameters (delta optical density) at both times.

In the context of this experimental section, the parameters or steps which were specifically used or carried out to obtain the presented results are as follows:
a. TF (tissue factor) and PPL (phospholipids) are mixed with an activator of the intrinsic pathway, FXIa, and tPA (tissue plasminogen activator), at the following concentrations: 0.5 pM, 4 μM, 100 pM, and 0.13 μg/mL final test.
b. The mixture is incubated at 37° C. then the calcium ion-based triggering reagent is added to the mixture, 50 μL in 102 mM final reagent (17 mM final test), to initiate formation of the fibrin clot.
c. The change in optical density is read dynamically (every 2 seconds) at a single wavelength, at 540 nm and for a duration of 1986 seconds (33 minutes).
d. The database is composed of all the deltas of the optical density (DOD) at the different measurement times and for each of the method parameters (Tmax; T09D—90% of the max DOD; TL—50% of the max DOD—max DOD is a measure of optical density).
e. These results are analyzed using quadratic discriminant analysis to determine the % of correct classification at t1 and t2.
f. This database is used as a training database which is trained on at least 70% of the overloaded samples selected randomly, and as a test database for the remaining overloaded samples (30%), then as a validation database for the patient samples.

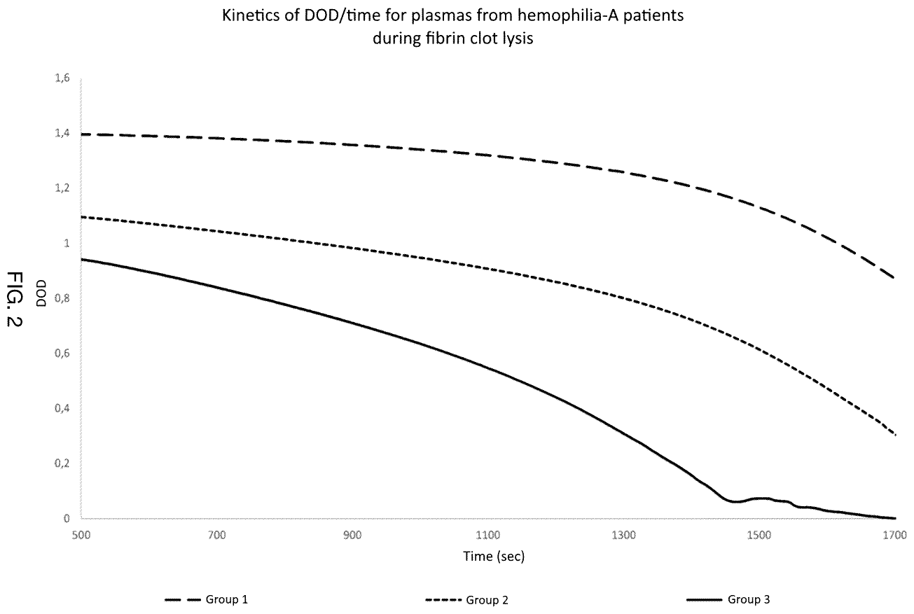

FIG. 2 shows what typical fibrin degradation profiles look like for three different hemophilia-A plasmas. FIG. 2 shows that the DOD (delta optical density) parameters at times 1 and 2 vary according to the hyper/hypo-fibrinolysis of the plasma of a hemophilia patient.

### B. Study of all Samples and Training Database

The set of samples may include hemophilia plasmas and in particular hemophilia-A plasmas with or without inhibitors, and samples from patients treated on demand or preventively. The method is possible with all commercial treatments and preferably with Advate®, Elocta®, and Jivi®. The reasons why these different treatments do not induce any bias into the analysis concerning the samples used, are linked to the marketing authorizations for said drugs with different mechanisms of action (rFVIII and “long-acting FVIII”).

In particular:
a. Advate® (Takeda) is a recombinant molecule that is currently a market leader; https://www.fortunebusinessinsights.com/industry-reports/hemophilia-drugs-market-100068
b. Elocta® (Sanofi) and Jivi® (Bayer) are “Long-Acting FVIII”. Elocta® is the only product with a long half-life that is authorized in France and involves a fusion with an FC immunoglobulin fragment; around 20% of the market: https://www.lesechos.fr/2018/01/sanofi-fait-son-entree-sur-le-marche-de-lhemophilie-982460. Jivi® is a PEGylated (polyethylene glycol) molecule from Bayer, a molecule which is considered representative of the three PEGylated molecules authorized for sale.

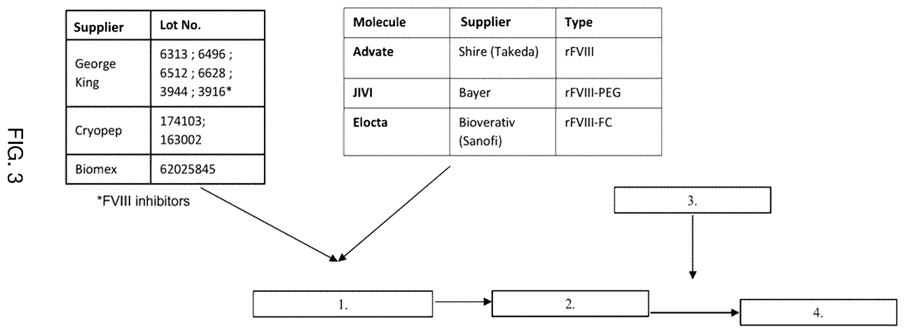

The data reported here are based on the use of 9 severe hemophilia-A patients overloaded with three FVIII molecules (Advate®, Elocta®, and Jivi®) (FIG. 3). Samples from hemophilia-A patients before and after treatment with Jivi® or Elocta® (the type of treatment was carried out blinded) were analyzed.

The training database was created using severe hemophilia-A plasma (<1%) overloaded with molecules at different concentrations. A total of 250 samples (and results) from non-overloaded and overloaded patients were analyzed as a training and testing group. Then 47 additional patient samples were analyzed as a validation group. In general, a training sample usually includes between 50% and 80% of the data and a test sample includes between 20% and 40% of the data, which is or may be separated randomly. Thus, for the present study, a total of 250 overloaded and non-overloaded patient samples were analyzed as a training (70%) and testing (30%) group and then 47 additional patient samples were used for the model validation.

Plasmas from overloaded patients were obtained following this protocol:
Several 1 mL aliquots of patient plasma are thawed for 5 minutes at 37° C. and mixed together. 100% FVIII overload is obtained while complying with a maximum plasma dilution of 2%. The FVIII level obtained after plasma overload is evaluated using an automated chromogenic assay sensitive to FVIII.
After adjusting the level to 100%, the plasma ranges are obtained by diluting the 100% level in the plasma down to 0% so as to obtain all of the following levels: 0%; 1%; 2%; 5%; 7%; 10%; 15%; 20%; 60%; 80%; 100% FVIII. Note: some intermediate levels (e.g. 15; 80%) were not manufactured for all overload ranges.

After a minimum of 24 hours of freezing at −80° C., each range is tested with the Coagulation-Lysis test.

The plasmas of hemophilia-A patients tested for the invention are collected according to two pharmacokinetic profiles: pre-dose and 0.25 h, 0.5 h, 1 h post-dose. Each sample is tested using an automated chromogenic assay sensitive to FVIII and the Coagulation-Lysis test.

Statistical analysis of all samples is carried out using statistical software, preferably the Minitab 18 and R software.

### C. Statistical Analysis on DOD (Delta Optical Density) Parameters at Two Times

Statistical analysis is carried out on the DOD (delta optical density) parameter at two times.

Initially, differences in the lysis profile were observed (FIG. 2).

Then, three groups were statistically highlighted for the two DOD (delta optical density) parameters at two times, in centered and normalized hierarchical analysis. Hierarchical analysis (centered and normalized)—or observation in groups—was also used to classify the training data, which were confirmed by quadratic discriminant analysis.

Lastly, the inventors obtained a correct classification rate of 100% by using a quadratic discriminant analysis as a training model, and 99% (3 errors) by the normalized K-means method (analyses carried out in Minitab)—the results obtained by the normalized K-means method are not represented in the Figures of this patent application, only the results obtained by quadratic discriminant analysis. Furthermore, the correct classification rate of 100% obtained by quadratic discriminant analysis was obtained with samples where factor FVIII was greater than or equal to 1.5% (10 errors otherwise, if 0% levels are included), when all data (250 results+47 patients) are considered.

The classification group is determined using the two DOD (delta optical density) parameters. For the turbidimetric method used, the times are defined within the ranges of [700-900] sec for time t1 and [1100-1400] sec for time t2. Preferably, time t1 was defined at 750 sec and time t2 was defined at 1400 sec. More precisely, the results shown in the Figures were obtained with t1=750 sec and t2=1400 sec. These parameters were selected in accordance with the method for the optimal choice or verification of these parameters, explained in this description. In fact, the range of [700-900] sec is the range of results that showed the best differentiation when all results were taken into account with only one parameter. The range for time t2 was defined in combination with the results obtained at time t1.

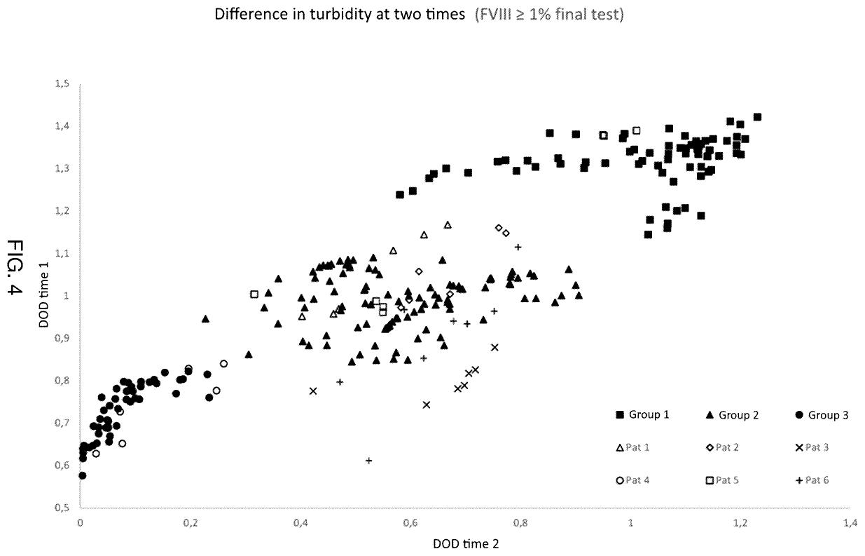

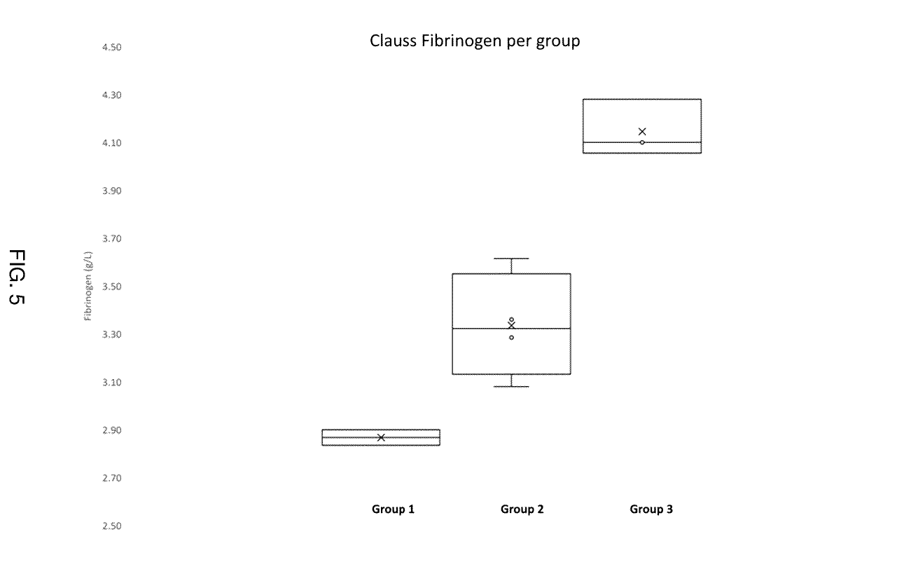

Each patient is classified with these parameters, measured at two times, according to the lysis, compared to the training group constructed using quadratic discriminant analysis under the same conditions (FIG. 4: Results by grouping).

Note: Patient no. 5 did not show the same classification based on his pharmacokinetic profiles for Jivi® or Elocta®. He moved from group no. 1 to group no. 2. This difference is linked to the basal level of the fibrin clot (DOD time t1). In this regard, we specify here that the assignment to a group may first take place by an unsupervised learning method. Visual inspection of the results can prove useful in certain specific cases (example here of patient no. 5). FIG. 4 shows that patient no. 5 is indeed classified into both groups no. 2 and 3 (empty squares in the legend). Indeed, patient no. 5 was sampled at two different times. In the first case, his fibrinogen was at 3 g/L with a low risk of bleeding. In the second case, he undoubtedly had had a bleeding event which increased his inflammation, hence the increase in fibrinogen to 4.2 g/L, which explains his change in classification. However, this particular case does not interfere with the general presentation of this patent application, taking into account the risks of error inherent to the type of method presented herein, which moreover can be calculated, as detailed herein.

Patients in group no. 1 (FIG. 4) have a high basal level of fibrin clot (DOD at time t1>1.14). This was verified by measurement of the fibrinogen level, which is higher than the normal region (4.0 to 5.0 g/L). In addition, patients in group no. 1 have significantly faster lysis (from −1.2 mDOD/sec to −2.5 mDOD/sec).

Patients in group no. 2 (FIG. 4) have a moderate basal level of fibrin clot and moderate degraded fibrin level. This group constitutes the reference group. It has a lysis time of between 1100 sec and 1900 sec, and a lysis slope of between −2.2 mDOD/sec and −0.7 mDOD/sec.

Patients in group no. 3 (FIG. 4) have a reduced basal level of fibrin clot and a reduced degraded fibrin level (a reduced DOD at times t1 and t2, respectively <1.14 and <0.26). This was verified with measurement of the lysis time (TL), which is significantly reduced for this group (from 840 sec to 1300 sec).

### Additional Analysis: Random Forest

We performed the analysis with two parameters per tree and 3000 trees.

On the training data, we obtained a correct classification rate of 100%, and 68% (13 errors) on the validation data (patients) compared to the reference classification. These values concern FVIII levels >1.5. The first value corresponds to the database of overload results and the second to patient results. It is also possible to group these values into a single figure of 95% correct classifications.

### Additional Analysis: Logistic Regression

We performed the analysis in multinomial logistic regression, taking group 2 as a reference (the analysis is similar when taking groups 1 or 3 as a reference).

On the training data, we obtained a correct classification rate of 100%, and 93% (3 errors) on the validation data (patients) compared to the reference classification. These values concern FVIII levels >1.5. The first value corresponds to the database of overload results and the second to patient results. It is also possible to group these values into a single figure of 99% correct classifications.

### Additional Analysis: Other Classification Techniques Using Artificial Intelligence (Principal Component Analysis or Neural Network)

With the aim of validating the partitioning obtained at DOD t1 and DOD t2, or of identifying whether another multivariate statistical method using all of the fibrinolysis kinetics would be more precise for classifying patients, two other classification techniques using Artificial Intelligence on the entirety of the lysis kinetics (starting from Tmax) of samples having a correct classification rate of 100% in quadratic discriminant analysis was implemented:
Principal component analysis (PCA) and application of a hierarchical method
A backpropagation neural network (autoencoder) and application of a hierarchical method
Application of the hierarchical method may (or may not) be preceded by t-SNE (t-distributed stochastic neighbor embedding) in order to represent in two dimensions the transformation of the data obtained by PCA or by the autoencoder.

### Conclusion (Results not Shown)

The statistical approach (PCA) and the learning approach (autoencoder) made it possible to extract a greater amount of information from the fibrinolysis kinetics, but the hierarchical methods applied to their output both converged towards similar partitionings (99% and 97% correct classifications compared to the method described respectively) to that obtained in quadratic discriminant analysis on DOD t1 and DOD t2. DOD t1 and DOD t2 parameters therefore appear to be sufficiently discriminating for effectively classifying patients into the different groups without needing to have access to the entire kinetics of fibrinolysis. This observation corroborates the concept underlying the present invention, which minimalistically exploits the parameters DOD t1 and DOD t2, hitherto unused for classification.

### D. Validity of the Training Model

According to one aspect, a minimum amount of FVIII is needed in the analyzed sample in order to sensitize the Coagulation-Lysis test method. However, the proposed classification is then, in itself, independent of the FVIII level and of the molecule used. Indeed, in the training database used, as the same plasmas have been overloaded at different concentrations and the “classification” is patient-dependent, it is possible to predict the ranking of the patient for all concentrations tested and in particular the 0% concentration.

Note that, as hemophilia patients are generally treated for their condition by administering a missing factor, the case where a tested sample does not meet the minimum FVIII requirement would be the exception rather than the rule in the field of the invention. Where appropriate, an exogenous contribution of missing factor to the sample to be tested may be provided for during implementation of the method according to the invention.

In the presence of all samples (including levels <1.5% and levels ≥1.5%, i.e. population N×0), an overlap of groups no. 3 and no. 2 was observed (FIG. 6). The level of “overlap” using quadratic discriminant analysis on the entire database (when 0% levels are included) was determined to be 97% (10 misclassifications) compared to the reference classification composed of the initial classification carried out with concentrations ≥1.5%—the overlap here is evaluated in terms of “number of correct classifications” compared to the initial classification carried out with concentrations ≥1.5%. In this regard, the initial classification was made using an unsupervised learning method with a factor FVIII level >1.5%. By extension, the 0 levels coming from the same patients were classified in the same groups.

One can see that if sensitization (provided by the study of levels ≥1.5%) made it possible to achieve 100% correct classifications and to eliminate the 10 classification errors (false positives), observation of results including 0% levels of Factor FVIII possibly show that patients in group no. 2 can be classified as no. 3 when they have a very low amount of Factor FVIII found in the corresponding samples. This remains of interest in identifying a “trough level” for Factor FVIII for group no. 2 and possibly in adjusting the FVIII replacement therapy in relation to this trough level. Therefore, the present invention is not limited to use on samples with a Factor FVIII level greater than or equal to 1.5%.

Furthermore, a limit to be set on the error rate mainly depends on the seriousness of the phenomenon studied. For example, according to one particular embodiment, one can consider the goal to be limiting the number of patients belonging to group 3 who are put in group 2. In fact, the patients belonging to group 3 have insufficient treatment: it can therefore be considered a group of interest to be identified as a priority.

According to one aspect of the invention, a result returned by discriminant analysis or another type of analysis can be evaluated by the “Number of correct classifications”, a goal of 90%, 91%, 92%, 93%, 94%, 95%, 96%, 97%, 98%, 99%, or 100% correct classification being a criterion for success of the test.

In the current case, the number of correct classifications can conventionally be obtained from the sensitivity/specificity measurement derived from a contingency table, as in the case of ROC curves. Here, the number of correct classifications was obtained from the sensitivity/specificity measurement derived from the contingency table shown in Table 1.

Based on data including 0% levels of Factor FVIII, the contingency table is as follows (Table 1):

TABLE 1
Classification summary

| Placement in | Actual group | | |
|---|---|---|---|
| group | 1 | 3 | 2 |
| 1 | 80 | 0 | 0 |
| 3 | 0 | 59 | 6 |
| 2 | 1 | 3 | 148 |
| Total number | 81 | 62 | 154 |
| N correct | 80 | 59 | 148 |
| Sensitivity | 0.988 | 0.952 | 0.961 |
| Specificity | 1.000 | 0.974 | 0.972 |

In this contingency table (Table 1): 6 results classified as Group no. 2 are found in group no. 3.

According to a specific but non-limiting embodiment of the invention, in particular where a Group 3 is apparent from the results as presented here, a result returned by discriminant analysis or another type of analysis can be evaluated by the “Number of correct classifications”, a goal of 90%, 91%, 92%, 93%, 94%, 95%, 96%, 97%, 98%, 99%, or 100% correct classification of patients in Group 3 being a criterion for success of the test.

### E. The Benefit of Grouping for Samples

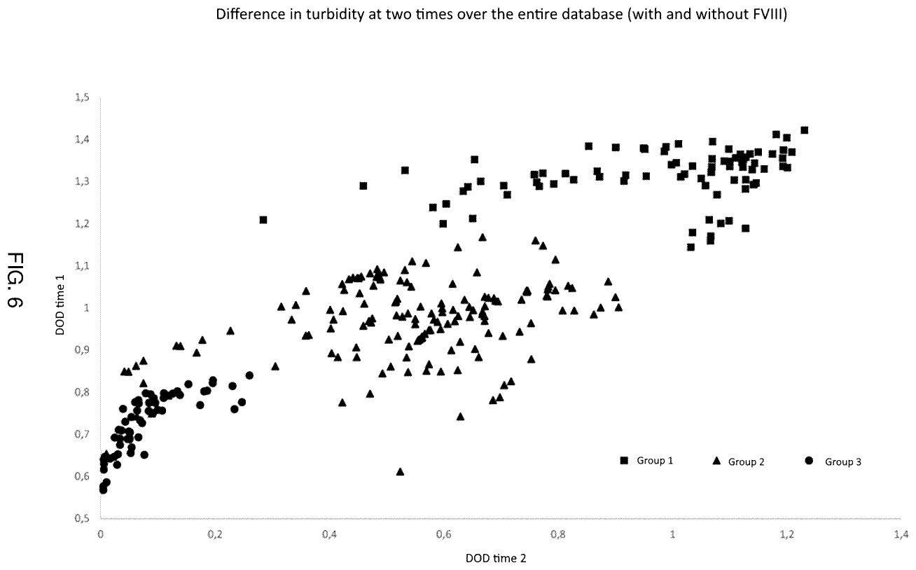

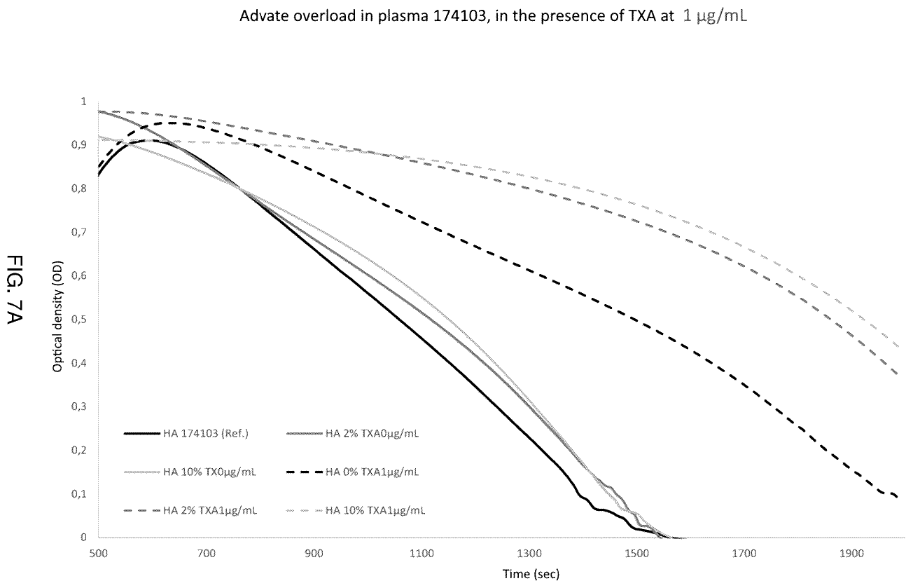

According to one aspect, the invention makes it possible to classify samples according to the groupings observed clinically (FIG. 7A and FIG. 7B).
Depending on their classification and grouping, plasma from hemophilia-A patients will react differently to the presence of FVIII and to TXA. This allows studying individual patients and the groups to which they belong, in order to identify abnormal lysis behavior and choose the right treatment. The test highlights the effect of TXA in reducing lysis. In the example presented here, it is the patients in group no. 3 with a reduced lysis time who require this treatment in combination with missing factor FVIII (FIG. 7).
The classification can also make it conceivable to personalize the trough level of a treatment with missing FVIII and/or TXA (“optimal trough level”) according to the assigned group.

Patient groups 1 and 2 (FIG. 7) do not require TXA but only require adjusting the treatment with the missing factor, with two adjustment subgroups.

More precisely, one can deduce that groups 1 and 2 would not need TXA, unlike group no. 3, as presented in the table below (Table 2):

TABLE 2

| Patient group | Treatment* | Comment |
|---|---|---|
| Group 3 | Replacement | TXA avoids increasing the |
| Patient at high risk of | therapy + TXA | doses of replacement |
| bleeding | | therapy |
| Group 2 | Patient comfort | Potential reduction in |
| Patient at low risk of | | replacement therapy |
| bleeding | | |
| Group 1 | Replacement | Potential increase in |
| Patient at moderate risk | therapy | already prescribed doses |
| of bleeding | | |

*Preventive: Advate ®: 20 to 40 U of FVIII/kg every 2 to 3 days
Novoeight ®: 20 to 40 U of FVIII/kg every 2 days up to 50 U 3 times/week; in the event
of bleeding, every 8 hours to every 2 days
Hemlibra ®: target population 1410 to 1880 patients, once/week or every 2 weeks for 4 weeks or every 4 weeks (HAS 2019).

### F. Study of Phenotyped Patients

Based on data from phenotyped patients, also presented are results for patient samples which are different from the aforementioned 47 additional patient samples, which have been analyzed.

### 1. Characteristics of Samples

a. 15 samples from 12 hemophilia-A patients: severe (8), moderate (2), and minor (2)
b. Treated on demand (3), preventively (7), or untreated (2)
c. Distribution of commercially available molecules, by sample:
   Recombinant FVIII: Advate® (2), Novoeight® (5), Kogenate® (2);
   Plasma-derived FVIII: Factane® (2);
   Recombinant FVIII with extended half-life: Elocta® (2)
d. The annual bleeding rate (ABR) for these patients is: 0 (5), 1 (2), 3 (1), 5 (2), 12 (1), 24 (1)
e. One patient was excluded from the study because out of specification (turbid)

### 2. Study of Patients

As with the 47 patient samples mentioned above, the 14 new samples were tested according to the method described here.

This allowed classifying the samples according to the DOD (delta optical density) at times t1 750 seconds and t2 1400 seconds.

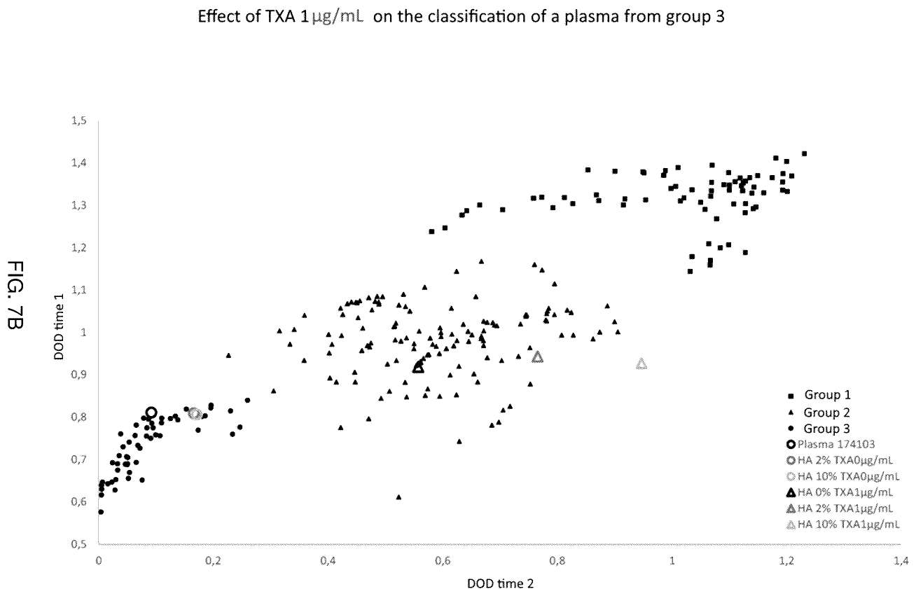

Note that samples taken from the same patient are classified in the same group (FIG. 8 and Table 3)

TABLE 3

| Treatment | N | ABR |
|---|---|---|
| Group no. 1 | | |
| Preventive | 3 | 7 (+/− 5) |
| On demand | 0 | NA |
| None | 1 | 24 |
| Total | 4 | 11 (+/− 9) |
| Group no. 2 | | |
| Preventive | 3 | 0 |
| On demand | 3 | 2 (+/− 2) |
| None | 1 | 0 |
| Total | 7 | 1 (+/− 2) |

No patient is classified in group no. 3, considered the group at high risk of bleeding. This can be explained by the fact that this group only represents 10% of patients (3/27) and therefore is not representative of 11 patients.

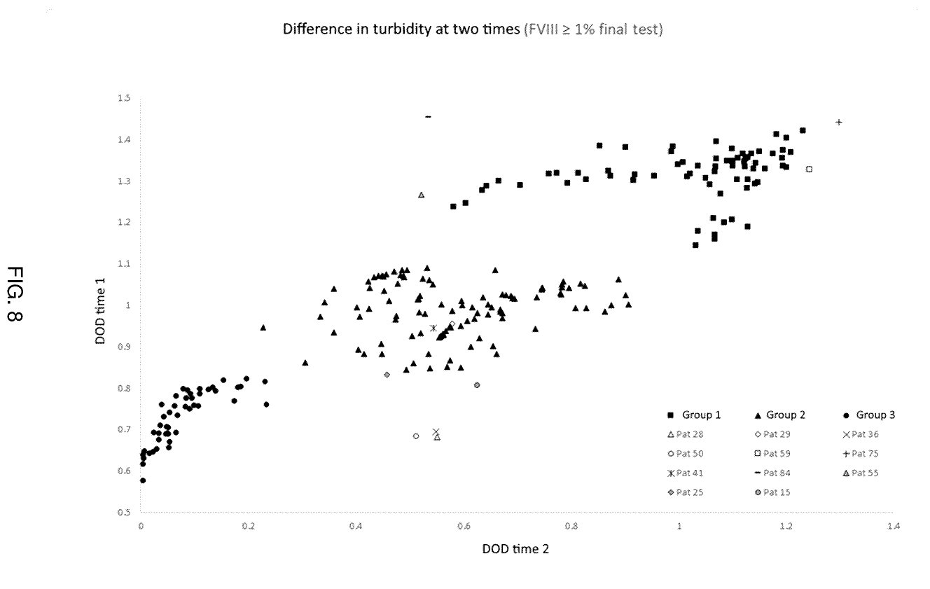

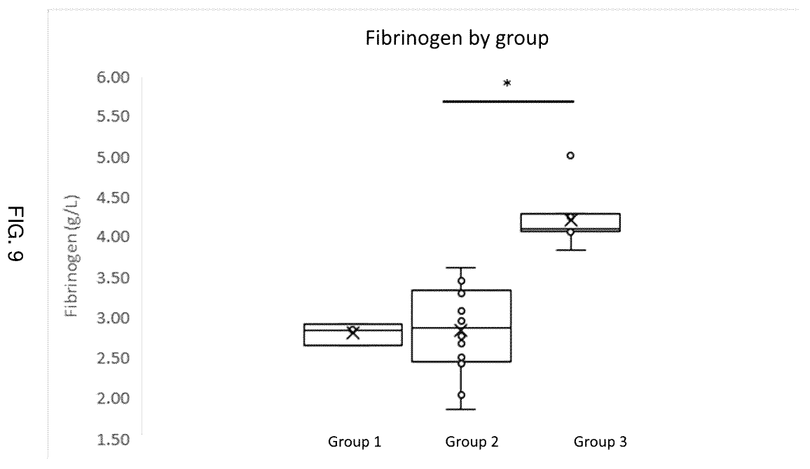

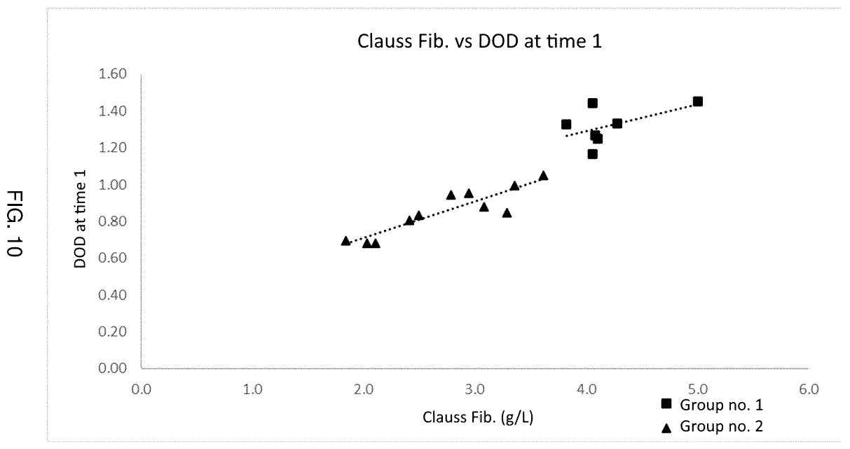

For patients classified in group no. 1, 4 out of 4 patients have an ABR≥3 (Table 3). This group is therefore at moderate risk of bleeding although treated preventively. These patients present regular bleeding, in which the key player is inflammation (Vulpen et al. 2018 DOI 10.1111/hae.13449.). This inflammation is expressed biologically by an increase in fibrinogen, as found in these patients (4.21 g/L group 1 vs 2.83 and 2.79 g/L groups 2 and 3 respectively, see FIG. 9) and also in the DOD at time 1 (FIG. 10). Although the fibrinogen level is much higher than that of the other groups and increases the lysis time (TL: 1668 sec vs 1460 and 1100 sec respectively), i.e. lysis occurs later, this does not protect them against bleeding because lysis takes place more quickly (slope at lysis time-Slope at TL: −1.92 mDOD/sec vs −1.40 and −1.13 mDOD/sec respectively) (FIGS. 11 and 12).

For patients classified in group no. 2, 5 out of 7 patients have an ABR=0 (Table 3). This means that these patients have a low risk of bleeding. The two patients (patients 25 and patient 41) whose ABR>0 are treated on demand, meaning their treatment is curative. These patients would benefit from a lower ABR by switching to personalized preventive treatment.

### 3. Conclusion

The above results are confirmed because the classification is reestablished in these new patients. The moderate bleeding of group 1 compared to the light bleeding of group 2 is explained. Group 3 remains the priority group at high risk of bleeding because it has both the lowest DOD at time 1 and a very shortened lysis time (1100 sec vs 1460 and 1668 sec).

### 4. Subsequent Clinical Data

At the end of the classification, two phenotyped hemophilia-A patients in group 2 who presented with bleeding changed treatment and were placed on preventive therapy. Patient 25, with an initial ABR of 5, was switched to “minimal prophylaxis” in March 2021, with the result of “unquantified clinical improvement in ABR”, and patient 41, with an initial ABR of 1, was switched to preventive therapy in 2020 with the result of an ABR of 0.

### G. Implementing a Viscoelastic Measurement Method

Using a ROTEM system on whole blood, here are the results obtained.

Switching to whole blood, the concentrations of the various activators were modified as follows:
The Coagulation-Lysis TF reagent was concentrated (2.2 pM TF and 8 UM Phospholipid)
FXIa was concentrated ×2 (200 pM FXIa)
The Coagulation-Lysis tPA reagent was diluted ×2 (0.07 μg/mL tPA)
The calcium buffer was not modified
The proportions (2/3) between samples and reagents were maintained for a larger final cuvette volume (340 μL).

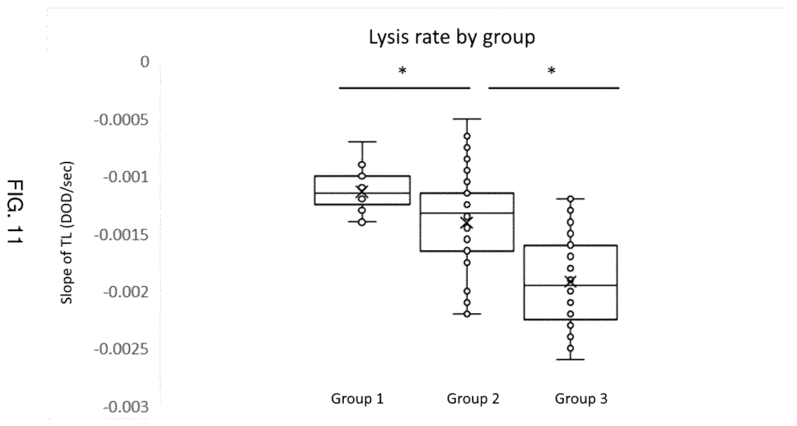

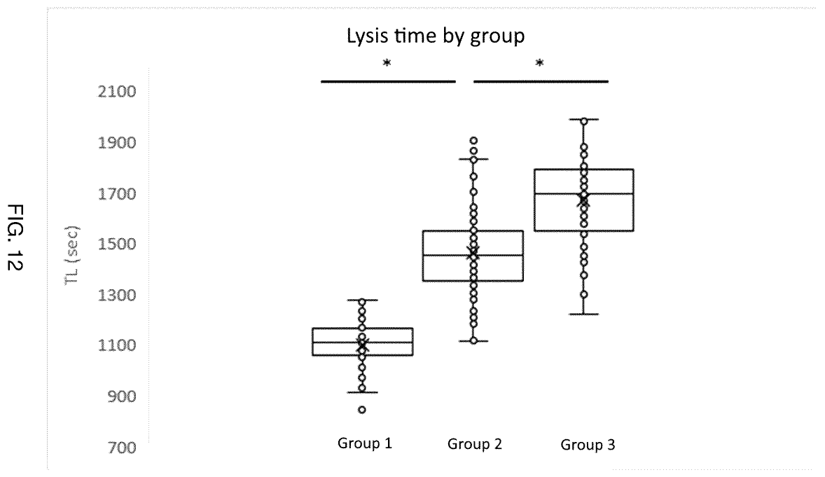

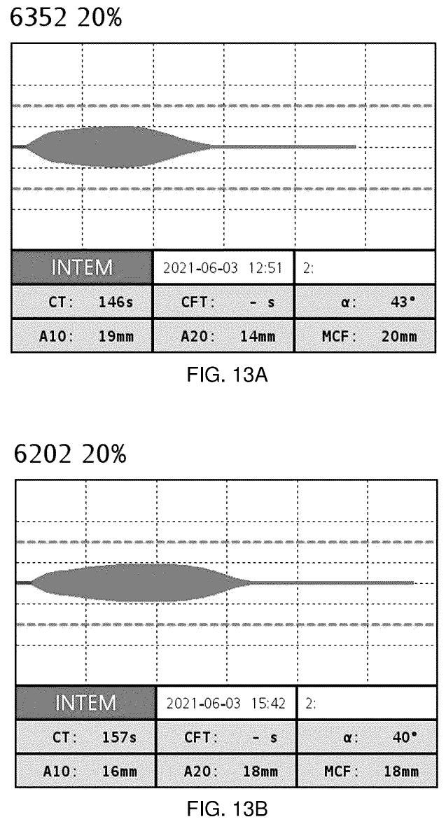

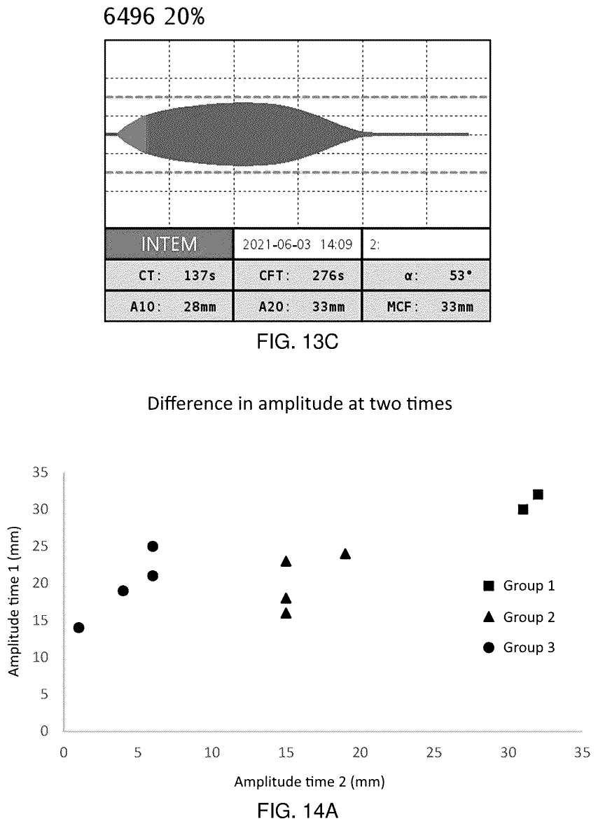

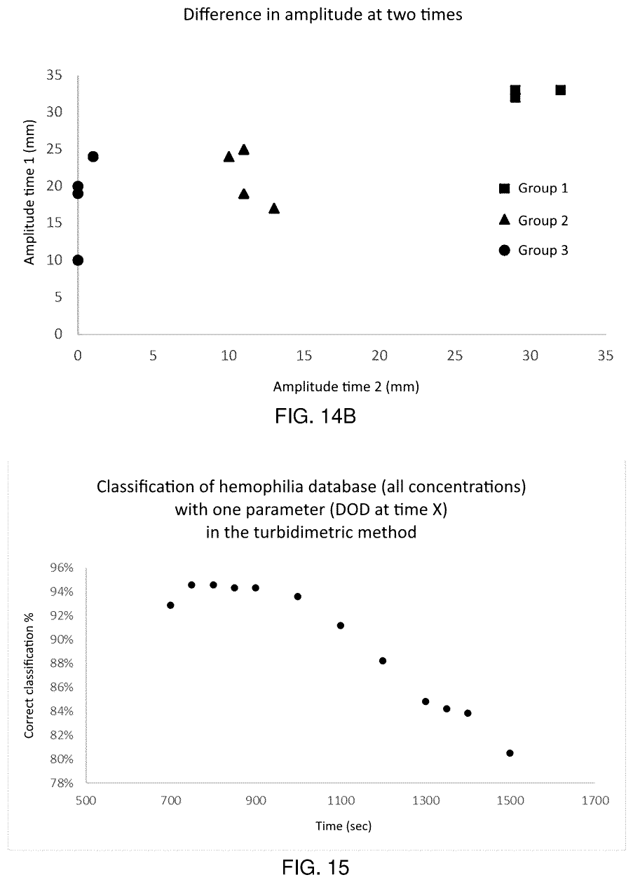

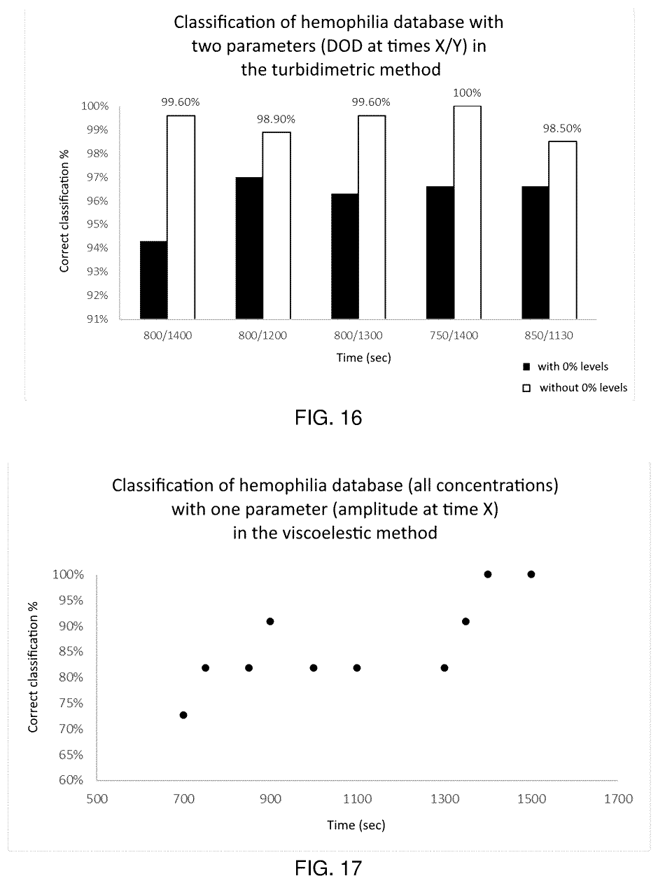

The inventors tested plasma from each group 1, 2, and 3, at different concentrations of FVIII under these conditions. The results of a test on the ROTEM system at a concentration of 20% FVIII (0.2 IU/mL) are shown in FIGS. 13A (Group 3), 13B (Group 2), and 13C (Group 1). In FIG. 13C, the change in grayscale appears automatically starting at 10 mm of amplitude; this allows calculating the ROTEM CFT (Clot formation time) parameter, not used in the present invention.

To do this, the inventors used the parameters yielded by the ROTEM A15 and A25 (15 minutes or 25 minutes after the coagulation time-CT) which correspond to parameters comparable to those used in the method of the invention described above, using an optical density measurement method (FIG. 14A). The inventors also reprocessed the results by taking t1 and t2 as fixed at 20 minutes and 30 minutes (FIG. 14B). This reprocessing of the results was carried out using image processing software on the image of the produced curve (software developed in-house or http://www.graphreader.com) because the ROTEM does not provide raw data for each time. It only gives the parameters A5, A10, A15, A20, A25, LY30, LY45, which are respectively 5 to 45 minutes after the coagulation time. However, according to the method presented here, the result is processed at a fixed time after the triggering with calcium. During this reprocessing, the inventors ensured beforehand that the results returned were the same as those from the ROTEM (+/−1 mm).

It follows that the method according to the invention can also be implemented using a viscoelastic measurement method with whole blood, in particular on a ROTEM system.

### H. Determination of a ROC Curve in Order to Evaluate the Sensitivity and Specificity of the Classification

As noted above, the number of correct classifications can be obtained conventionally, from the sensitivity/specificity measurement derived from a contingency table, as in the case of ROC curves.

By way of illustration, we wanted to evaluate an embodiment of a method for classifying hemophilia patients according to the present invention, employing the two parameters DOD t1 (750 seconds) and DOD t2 (1400 seconds). To do this, we processed the data with samples where the FVIII was greater than or equal to 1.5% and a correct classification rate of 100% was obtained in quadratic discriminant analysis. These data were compared to two thresholds obtained using ROC curves, in order to have a sensitivity of 100% in the samples for groups 1 and 3.

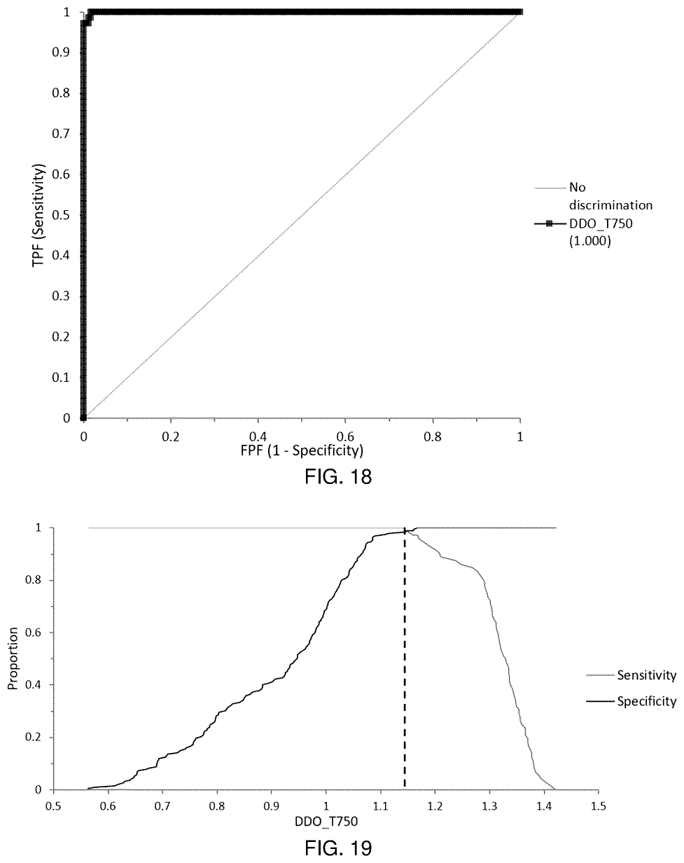

### 1. Threshold at DOD t1 (FIGS. 18 and 19)

The DOD t1 measurement allows us to include patients in group 1.
The threshold of 1.144 DOD was obtained for this parameter on the ROC curve, using the Analyse-it add-in.
A DOD t1 value above the threshold allows classifying the patient sample in group 1.
All samples from group 1 were correctly classified (100% sensitivity).
4 samples from group 2 were incorrectly classified in group 1 (specificity of 98%).

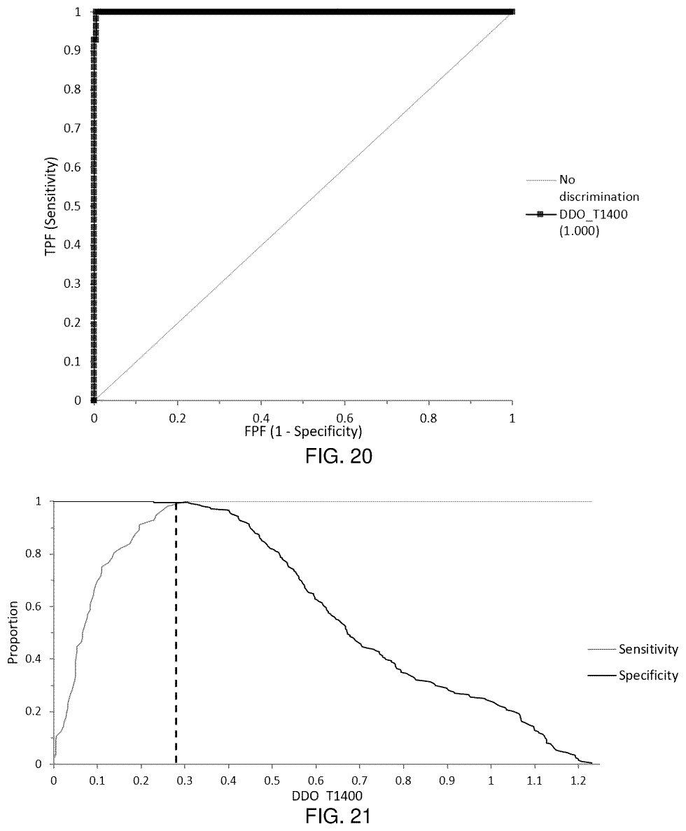

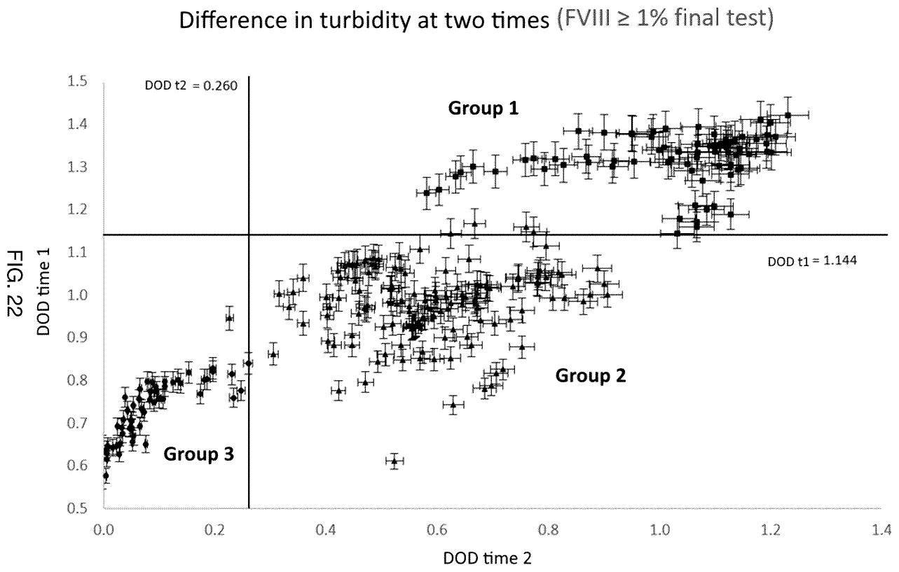

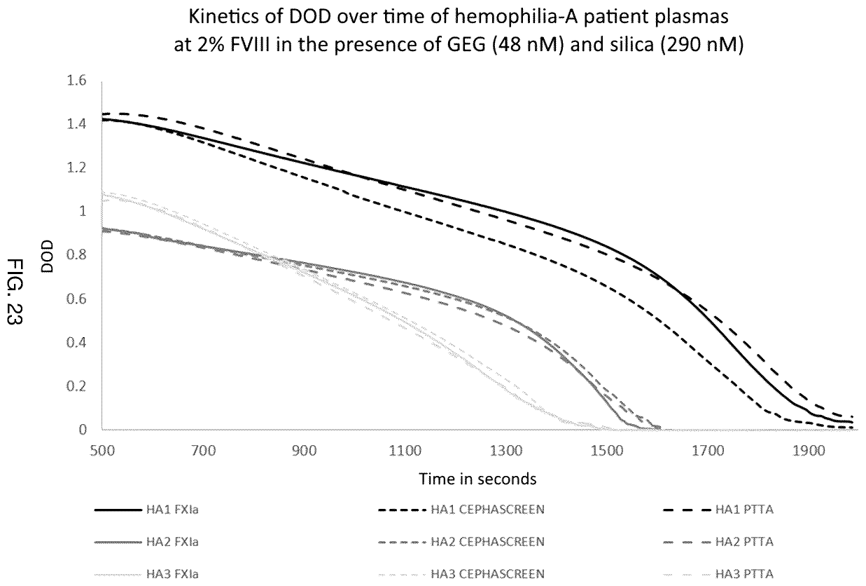

### 2. Threshold at DOD t2 (FIGS. 20 and 21)

The DOD t2 measurement allows us to include patients in group 3.
The threshold of 0.260 DOD was obtained for this parameter by the ROC curve, using the Analyse-it add-in.
A DOD t2 value below the threshold allows classifying the patient sample in group 3.
All patients in group 3 were correctly classified (100% sensitivity).
1 sample from group 2 was incorrectly classified in group 3 (specificity of 99%).

DOD values such that DOD t1 is below the threshold while at the same time DOD t2 is above the threshold, allows the patient sample to be classified in group 2.

### Conclusion

The two thresholds at DOD t1 and DOD t2 allowed us to have a correct classification rate of 98% with 5 classification errors in relation to the described method, and a sensitivity of 100% for groups 1 and 3 which seem to correspond to the groups at a higher risk of bleeding. These thresholds can be used, as this illustration shows, in parallel with a particular embodiment of the method described herein for classifying patient samples.

### I. Study of Intrinsic Activators in Turbidimetry

Matsumoto et al. 2009 showed that, within the framework of the method they describe, the optimal concentration of ellagic acid is 300 nM in association with 0.5 pM tissue factor (TF). As for He et al 2018, they propose triggering the coagulation reaction on ROTEM (viscoelastic method) in plasma with the Stago STA-PTT analyzer reagent diluted to 1.2×10-3 of the original dose in the presence of TF at 0.02 pM.

Our work has led to expanding the use of intrinsic activators in addition to Factor XIa (FXIa). The aim here is to verify the feasibility of the Lysis test independently of the activating reagent while remaining compatible with the turbidimetric measurement method at 540 nm.

### 1. Method of Analysis

The intrinsic activators in Stago reagents are ethylene glycol gallate at 23 mg/L (STA-Cephascreen) and silica at 20 g/L (STA-PTT analyzer).

As was done for the implementation of a viscoelastic method, turbidimetry tests were performed with a hemophilia-A patient from each group, overloaded with 0, 2, 20, 100% of Factor VIII (FVIII-Advate®). The coagulation reaction was initiated with ethylene glycol gallate (GEG) from 48 to 190 nM final test or with silica from 145 to 580 nM final test, without modifying the concentration of tissue factor which was kept at 0.5 pM final test for the two activators. These activators were added independently into the reagent under the same reference conditions as FXIa.

The aim is to determine the maximum difference in tolerance observed in parameters DOD t1, DOD t2, and TL, between FXIa and the intrinsic activators.

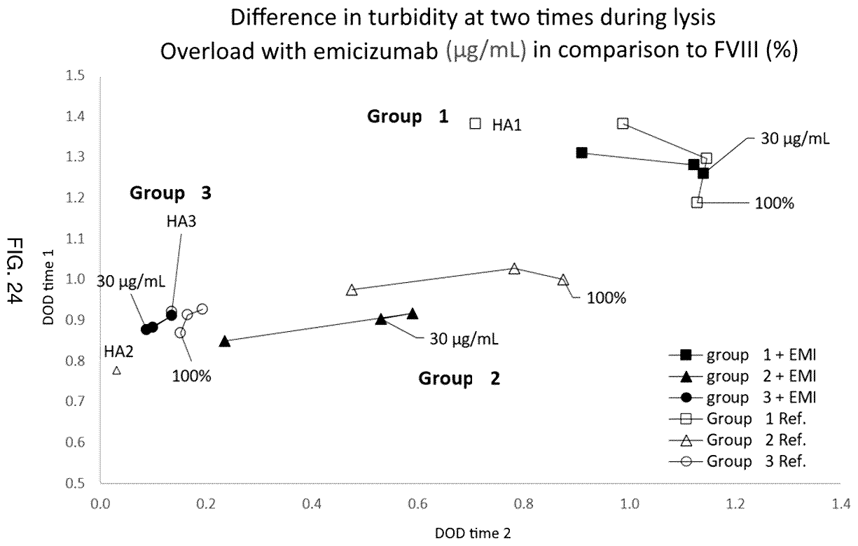

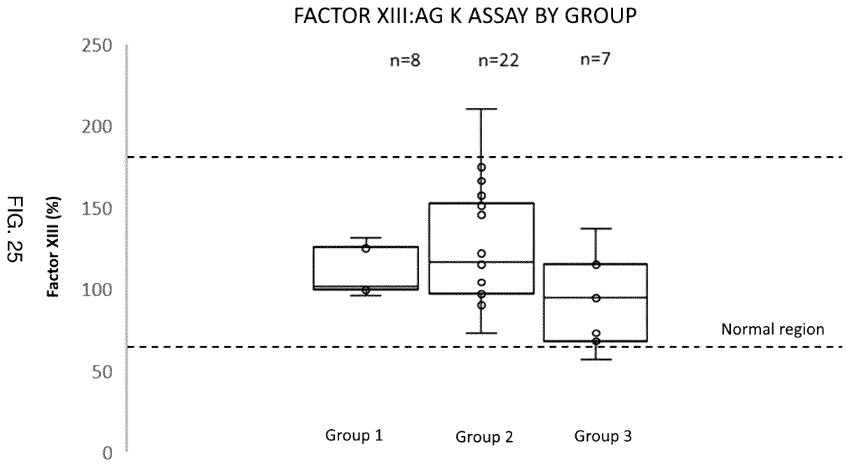

For clarity in the graphs of FIGS. 22 and 23, only the 2% concentration of FVIII is shown.

### 2. Results (FIGS. 22 and 23)

The final optimized test concentrations are 48 nM of GEG and 290 nM of silica.

At these concentrations, the maximum deviations accepted for all FVIII concentrations tested, for parameters DOD t1, DOD t2, and TL, compared to the reference method with FXIa, are:
−9% with GEG and +7% with silica for group 1,
+3% with GEG and +8% with silica for group 2,
+8% with GEG and +14% with silica for group 3.

After computer simulation of +3% median deviation observed over all groups and for all concentrations of FVIII, none of the 15 hemophilia-A patients in the patent changed their classification group compared to the reference condition of triggering with FXIa. Furthermore, the correct classification rate can be reduced, considering the overlaps between different errors per group. It is therefore possible, with any new activator, to determine that it is indeed possible to classify patients into groups with the same level of differentiation as with the reference activator FXIa.

### 4. Conclusion

FXIa can therefore be replaced by GEG at 48 nM or silica at 290 nM final test, while retaining [0.01 to 5.0 ] pM tissue factor final test. By extension, the applicability of the 300 nM concentration of ellagic acid, found in the bibliography, is anticipated to be favorable.

### J. Study of Therapeutic Molecules

### 1. Method of Analysis

As we did for the implementation of a viscoelastic method, we carried out turbidimetry tests where a hemophilia-A patient from each group is overloaded with different concentrations of therapeutic molecules of interest.

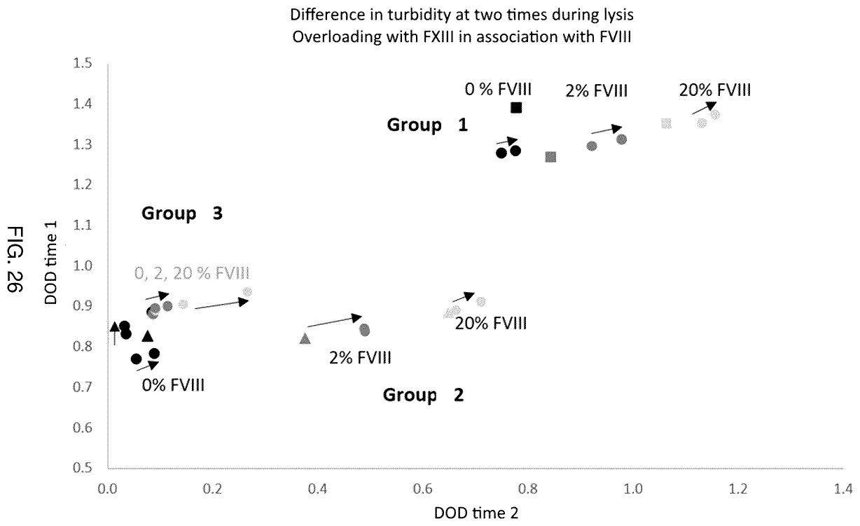

### 2. Bispecific Antibody, Example Emicizumab—FIG. 24

In hemophilia-A applications with or without inhibitors, bispecific antibodies are agents mimicking the action of FVIII by forming a complex with activated Factor IX (FIX) and Factor X in order to overcome the FVIII deficiency. They have the dual advantage of being administered via subcutaneous injections and having long half-lives. Emicizumab (Hemlibra®, Roche) is the commercial bispecific antibody of reference in the treatment of hemophilia A with and without inhibitors (Mahlangu et al. 2021).

Coagulation tests based on aPTT and human FVIII must be modified to measure their circulating amounts via specific calibrators and controls. These tests were not able to measure the hemostatic effects of these molecules (Lowe et al. 2020).

We wanted to test this type of molecule (below and FIG. 24) at concentrations of 0, 3, 15, and 30 μg/mL (Nougier et al. 2020) compared to the reference FVIII treatment (Advate®) at 0, 2, 20, and 100%.

Regardless of the patient groups, there is little change to DOD t1 via emicizumab overload. DOD t2 of groups 1 and 2 is increased up to 15 μg/mL of emicizumab, reflecting greater resistance to lysis, as observed with an increasing concentration of FVIII. No change in the group of overloaded hemophiliacs is observed. There is little change in parameters for the patient in group 3. An overlap in classification between the patient in group 2 and group 3 is observed at 3 μg/mL emicizumab.

With the method described, a similar evolution is observed between overloads with emicizumab and with FVIII regardless of the patient group, increasing the resistance to lysis of patients in groups 1 and 2.

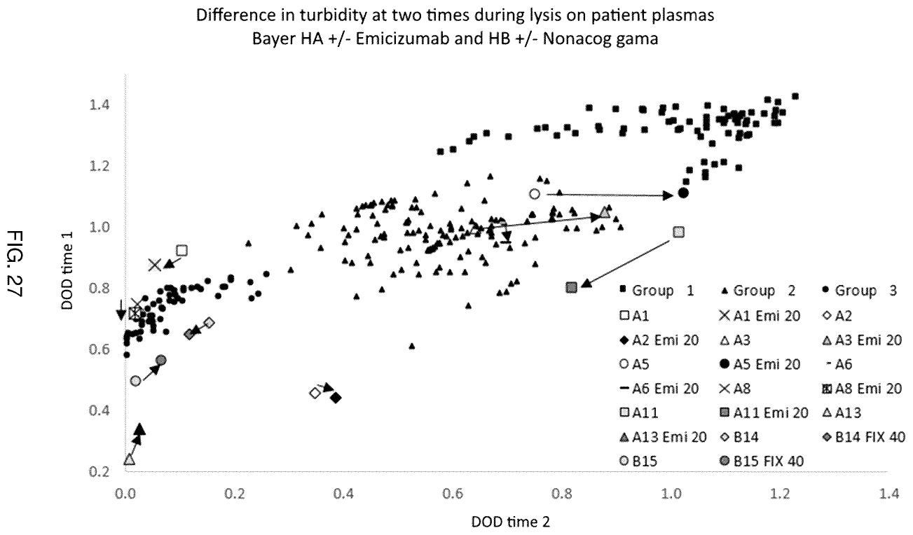

### 3. Factor XIII—FIGS. 25 and 26

Factor XIII (FXIII) or Catridecacog (NovoThirteen® NovoNordisk) is activated by thrombin. FXIIIa intervenes in the final phase of fibrin formation and fibrin polymerization in order to stabilize the clot. It provides a stable clot that is resistant to fibrinolysis. Co-administration of excess FVIII and FXIII has been shown to accelerate FXIIIa formation without increasing thrombin generation. Indeed, in the plasma and whole blood of hemophilia-A patients, co-treatment by FXIII with FVIII made it possible to increase the generation and quantity of cross-linked fibrin and to increase the weight of the clot (Beckman et al. 2018).

The level of FXIII was measured as antigen (FXIII: Ag) in all patients in the study, using the Factor XIII K Assay reagent. According to the kit instructions, the normal range of FXIII: Ag is [59-181] %. The average measured level is 117% for the three groups of patients (values ranging from 57 to 211%): 110% for group 1 (n=8), 127% for group 2 (n=22), and 92% for group 3 (n=7). One patient with a level below 59% was measured in group 3 at 57%. There is no significant difference between the three groups (ANOVA with Minitab 18 software: p>0.05)—FIG. 25.

One patient from each of the three groups was overloaded with increasing concentrations of FXIII (NovoThirteen®) and three levels of FVIII (Advate®—0, 2, and 20%):
Group 1: patient 6496 was overloaded with FXIII from 130% to 160 and 200%
Group 2: patient 62025845 was overloaded with FXIII from 120% to 150 and 200%
Group 3: patient 6352 was overloaded with FXIII from 90% to 130 and 170%

Note: for practical reasons, the overload was not adapted as a function of the amount of physiological FXIII: Ag (respectively 130, 120, and 90%) present in the three patient plasmas.

We observe (FIG. 26) that FXIII, mainly in combination with FVIII, increases the DOD t2 parameter of patients in the three groups. With 2% FVIII and >150% FXIII: Ag, DOD t2 increases from 9 to 34% compared to the condition without FXIII, which would result in greater resistance of the clot to lysis. Furthermore, a change of group is noted for the hemophilia-A patient in group 3. Indeed, with 20% FVIII and 170% FXIII: Ag, this patient moves from group 3 to group 2.

FXIII in combination with FVIII increases the resistance to lysis of patients from the three groups. In particular, it would seem that an excess of FXIII in association with FVIII would be beneficial for the patient representative of group 3. This could be confirmed with other patients in this same group.

4. Acquired Hemophilia a and Treatment with Susoctocog Alfa

We have shown with new patients, in point L. below, that the classification method described could be used with plasma from hemophilia-A or B patients with inhibitors. Acquired hemophilia A or B is a rare non-hereditary (non-genetic) bleeding disease due to the presence of antibodies directed against a coagulation factor (FVIII or FIX), which reduce its coagulant activity to levels comparable to the genetic disease.

An approved treatment for acquired hemophilia A is a recombinant porcine sequence factor FVIII: Susoctogog alfa (Obizur® Takeda). This molecule is indicated in the treatment of bleeding episodes in adult patients with acquired hemophilia due to antibodies which the patients have developed against FVIII. Its active ingredient, susoctocog alfa, shows little cross-reactivity with the anti-FVIII inhibitor of human origin. Its lower sensitivity to inactivation by the patient's autoantibodies makes this molecule capable of restoring hemostasis by replacing the inhibited FVIII.

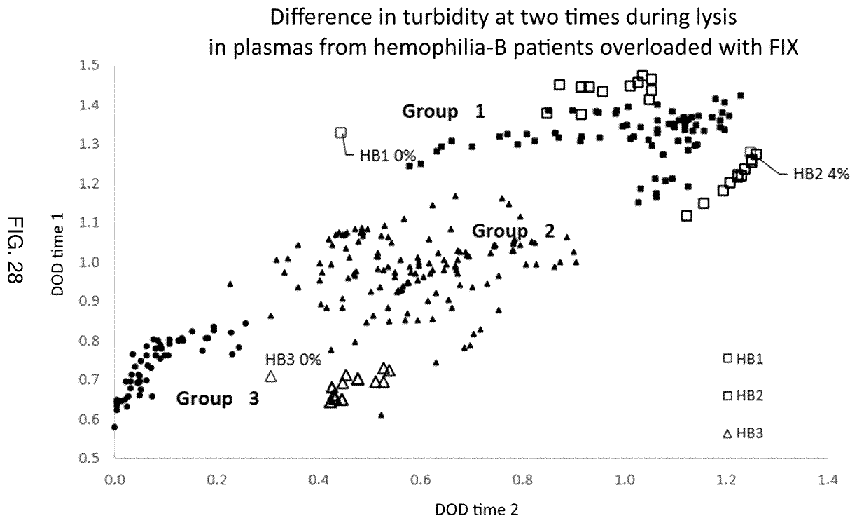

As part of the study of new molecules, we tested Obizur® on two severe hemophilia-A patients: a model patient from group 1 containing inhibitors (3916) and a patient from group 3 (6352)—FIG. 27: we thus show that the described method is feasible with severe hemophilia-A plasmas overloaded with Obizur®.

### K. Applicability to Hemophilia-B Patients

As with hemophilia A, hemophilia B is a genetic disease that affects circulating FIX levels. There are three forms of hemophilia B, whose classification is based on the plasma level of FIX:
Severe form: <1%
Moderate form: 1% to 5%
Minor form: 5% to 40%

Severe hemophilia B represents between 30 and 40% of cases. Hemophilia B is less studied than hemophilia A in the literature because fewer patients are affected. Spontaneous bleeding is non-significantly less frequent (ABR=14.4 hemophilia A vs 8.63 hemophilia B) but the consequences are similar between the two diseases (Dolan et al. 2018).

Treatment consists of replacing the missing FIX with a plasma derivative or a recombinant product. It can be administered after bleeding (on-demand treatment) or to prevent bleeding (preventive treatment). As with hemophilia A, the most common complication is the appearance of antibodies directed against the coagulation factor inhibiting its action (called inhibitory antibodies). However, inhibitors are much less common in hemophilia B (<5%) than in hemophilia A (˜30-50%). (Dolan et al. 2018).

### 1. Method of Analysis and Goals

Three hemophilia-B plasmas, two severe and one moderate (HB1 7394, HB2 150508, and HB3 150509) overloaded with three FIX molecules (Takeda Rixubis®, LFB Betafact® and NovoNordisk Rebinyn®) are assayed under the same reference conditions as those for hemophilia-A plasmas (TF, tPA, FXIa, turbidimetry).

The aim is to show that the classification method using DOD t1 and DOD t2 applies equally well to both hemophilia A and hemophilia B.

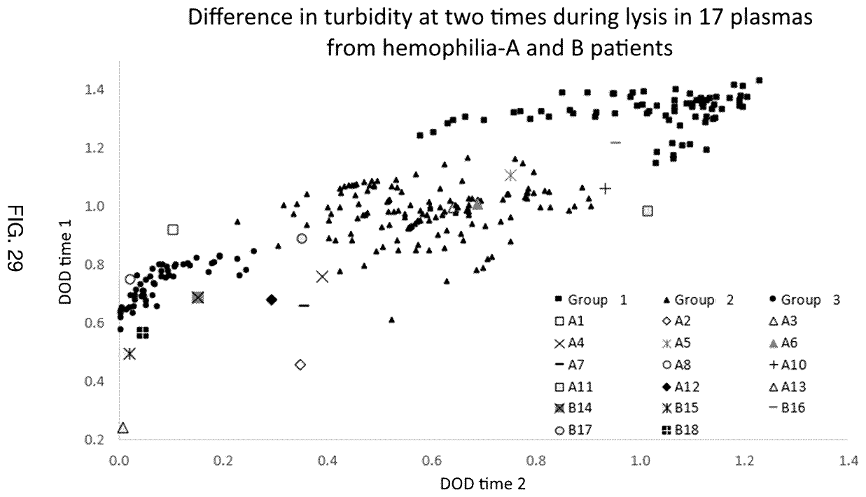

### 2. Results (FIG. 28)

The groups optimized for hemophilia A allow classifying two of the three hemophilia-B patients (Patient 1 in group 1 and patient 3 in group 2). Patient 2 (moderate) could not be classified due to the complete absence of fibrinolysis in the test's measurement duration of 1986 seconds. It is suspected that he is being treated with tranexamic acid or equivalent. The addition of different FIX molecules does not change the classification of these two patients.

The described classification method using the parameters DOD t1 and DOD t2 is therefore applicable with hemophilia-B plasmas.

### L. Applicability to a Different Patient Group

In addition to the 47 samples from 6 hemophilia-A patients presented above, other results were obtained on a previously unreviewed batch of 17 new hemophilia A and B patients, with and without inhibitors. The aim was to show that the classification method using DOD t1 and DOD t2 also applies to these new hemophilia-A and B patients.

### 1. Sample Characteristics

We received 17 patients with severe hemophilia A (denoted A1 to A13) and B (denoted B14 to B18):
12 hemophilia A: 2 patients with inhibitors; 10 patients without inhibitor
5 hemophilia B: 4 patients with inhibitors; 1 patient without inhibitor

### 2. Method

We assayed the 17 samples from patients either untreated or after overloading with a replacement molecule (Emicizumab for hemophilia A and Rixubis® for hemophilia B—“missing factor”) according to the method described herein. The presence of the replacement molecule makes it possible to avoid overlap between the observed groups when patients are untreated.

For certain hemophilia patients, the presence of inhibitors justifies the use of emicizumab. As this type of antibody does not exist for hemophilia B, 40% Rixubis® was used with 30 minutes of incubation at 37° C. to allow neutralization by the inhibitor:
8/13 hemophilia-A patients were able to be overloaded with emicizumab at 15 μg/mL, measured at 20 μg/mL
2/5 hemophilia B patients were able to be overloaded with 40% Rixubis® with a residual titer of FIX after neutralization of 1% (B14) and 21% (B15)
1 patient classified in group 3 (B18) could not be confirmed by overloading the missing factor due to insufficient volume.

The 27 samples (17 samples+10 overloaded samples) were classified using the same parameters as for hemophilia A: DOD t1 at 750 seconds and t2 at 1400 seconds.

### 3. Classification Results

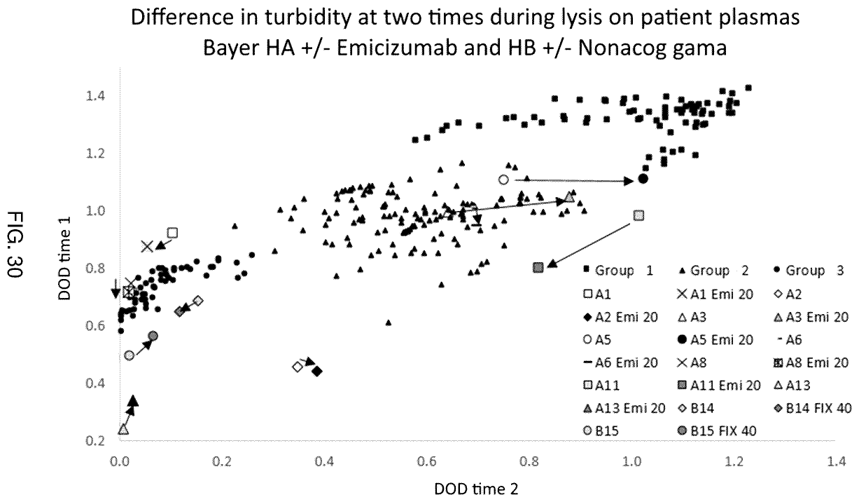

First, the 17 plasmas from hemophilia-A and B patients were analyzed without replacement molecules (“missing factor”)—FIG. 29: 94% of patient plasmas (16/17 patients) without the replacement molecule can be classified according to the described method: 1 plasma in group 1, 10 plasmas in group 2, and 5 plasmas in group 3. One patient (A1) changes classification (group 3 or group 2) depending on the training database or the threshold of the ROC curve; this needs to be confirmed by overloading with the missing factor (emicizumab).

Next, 10 of the 17 plasmas from hemophilia-A and B patients were analyzed with replacement molecules—FIG. 30: The patients' classifications remain unchanged in the presence of the replacement factor and according to the method described (or in ROC curves), in particular the replacement molecule made it possible to classify patient A1 according to the method described.

The classification method using the parameters DOD t1 and DOD t2 is applicable to a different batch of plasmas from hemophilia-A or B patients, with or without inhibitors and with or without a replacement molecule.

### INDUSTRIAL APPLICATION

These technical solutions can be applied in particular in the field of monitoring hemostasis via dedicated devices, or in the clinic with regard to patient monitoring.

The present disclosure is not limited to the examples described above solely by way of example, but encompasses all variants conceivable to those skilled in the art in the context of the protection sought.

### LIST OF CITED DOCUMENTS

### Patent Documents

For all practical purposes, the following patent document is cited:
WO 2016/012729 (publication number)

### Non-Patent Literature

For all practical purposes, the following non-patent elements are cited:
Matsumoto et al. 2009 Int J Hematol, A modified thrombin generation test for investigating very low levels of factor VI11 activity in hemophilia A, 10.10071˜12185-009-0450;
Tarandovskiy et al. 2013 Thrombosis Research, Investigation of the phenotype heterogeneity in severe hemophilia A using thromboelastography, thrombin generation, and thrombodynamics, 10.1016/j.thromres.2013.04.004;
Leong et al. 2017 Research and practice in thrombosis and haemostasis, Clot stability as a determinant of effective factor VIII replacement in hemophilia A, 10.1002/rth2.12034;
He et al. 2018 Thrombosis Research, A ROTEM method using APTT reagent and tissue factor as the clotting activators may better define bleeding heterogeneity in moderate or severe haemophilia A, 10.1016/j.thromres.2018.09.041;
Dargaud et al. 2017 Haemophilia, Individual thrombin generation and spontaneous bleeding rate during personalized prophylaxis with Nuwiq® (human-cl rhFVIII) in previously treated patients with severe haemophilia A, 10.1111/hae.13493;
Chitlur 2012 Thrombosis Research, Challenges in the laboratory analyses of bleeding disorders, 10.1016/j.thromres.2012.03.011;
Tripodi et al. 2019 Clinical Chemistry, Advances in the Treatment of Hemophilia: Implications for Laboratory Testing, 10.1373/clinchem.2017.284356;
Aghighi et al. 2019 Research and practice in thrombosis and haemostasis, Global coagulation assays in hemophilia A: A comparison to conventional assays, 10.1002/rth2.12295;
Tiede et al. 2020 Haematologica, Factor VIII activity and bleeding risk during prophylaxis for severe hemophilia A: a population pharmacokinetic model, 10.3324/haematol.2019.241554;
Abrantes et al. 2019 Haematologica, Relationship between factor VIII activity, bleeds and individual characteristics in severe hemophilia A patients, 10.3324/haematol.2019.217133
Delavene et al. 2020: Haemophilia, A new paradigm for personalized prophylaxis for patients with severe haemophilia A. DOI: 10.1111/hae. 13935.
Mahlangu et al. 2021: Haemophilia Wiley, Emicizumab state-of-the-art update. DOI: 10.1111/hae. 14524
Lowe et al. 2020: Haemophilia Wiley, Effects of Emicizumab on APTT, FVIII assays and FVIII Inhibitor assays using different reagents: Results of a UK NEQAS proficiency testing exercise. DOI: 10.1111/hae.14177
Dargaud et al. 2018: Haematologica, Use of thrombin generation assay to personalize treatment of breakthrough bleeds in a patient with hemophilia and inhibitors receiving prophylaxis with emicizumab. DOI: 10.3324/haematol.2017.185330
Nougier et al 2020: Haematology, Emicizumab treatment: Impact on coagulation tests and biological monitoring of haemostasis according to clinical situations (BIMHO group proposal). DOI: 10.1111/ejh.13490
Beckman et al. 2018: JTH, Factor XIII cotreatment with hemostatic agents in hemophilia A increases fibrin a-chain crosslinking, DOI: 10.1111/jth. 13887
Dolan et al. 2018: Blood review, Haemophilia B: Where are we now and what does the future hold? http://dx.doi.org/10.1016/j.blre.2017.08.007

1. Method for the in vitro measurement of fibrin clot degradation on the basis of a curve of the lysis of the fibrin clot over time in a blood or plasma sample previously obtained from a patient who may have a deficiency in at least one coagulation factor, the method comprising the following steps:
a. mixing the sample previously obtained from said patient with a composition of reagents comprising tissue factor, phospholipids, tPA, and one or more activator(s) of the intrinsic coagulation pathway, in particular one or more of these activators chosen among: ellagic acid, ethylene glycol gallate, silica, FIXa, FXIa, and FXIIa, and optionally one or more coagulation factors that are missing in the patient, in particular chosen among: FVIII, FIX, FXI, a bispecific antibody such as emicizumab; then
b. incubating the mixture obtained in a., then
c. triggering coagulation by adding calcium ions to the mixture incubated in step b. in order to allow a fibrin clot to form in the mixture, then allowing lysis of the formed clot, and
d. measuring the degradation kinetics of the fibrin clot during lysis in step c., and
e. determining a basal level value of the fibrin clot at a predetermined time t1, time t1 being chosen to be between the Tmax and TL of the curve of the lysis of the fibrin clot, and determining a degraded level value of the fibrin clot at a predetermined time t2 that is subsequent to time t1, time t2 being chosen to be between 300 to 900 seconds after t1.

2. Method according to claim 1, wherein, in step a:
i. The tissue factor is present in the composition of reagents in an amount such that the final concentration of tissue factor in the mixture on which the kinetics measurement is carried out in step d. is between 0.01 and 8.0 pM, or between 0.01 and 5.0 pM, or between 0.5 and 5.0 pM,
ii. The phospholipids are present in the composition of reagents in an amount such that the final concentration of phospholipids in the mixture on which the kinetics measurement is carried out in step d. is between 1 and 10 μM, or between 3 and 7 μM,
iii. The activator of the intrinsic coagulation pathway, in particular one or more chosen among: ellagic acid, ethylene glycol gallate, silica, FIXa, FXIa, and FXIIa, is present in the composition of reagents in an amount such that the final concentration of intrinsic activator in the mixture on which the kinetics measurement is carried out in step d. is between 1 pM and 600 nM, or between 10 and 200 pM,
iv. The tPA is present in the composition of reagents in an amount such that the final concentration of tPA in the mixture on which the kinetics measurement is carried out in step d. is between 0.01 and 5 μg/mL, or between 0.1 and 2 μg/mL,
The coagulation factor that is missing in the analyzed patient, in particular one or more chosen among: FVIII, FIX, FXI, is present in the mixture on which the kinetics measurement is carried out in step d. at a concentration between 0 to 2.0 IU/mL.
v.

3. Method according to claim 1, wherein, in step b., the incubation of the mixture obtained in a. is carried out between 2° and 39° C., for 2 to 10 minutes, then calcium ions are added to the incubated mixture in an amount allowing a final concentration of calcium ions that is between 5 and 25 mM.

4. Method according to claim 1, wherein the sample:
is an undiluted sample of whole blood or plasma, and/or
has a volume of between 5 μL and 500 μL.

5. Method according to claim 1, wherein:
a. If the sample is a whole blood sample, t2 is chosen to be 650 seconds after t1, and
b. If the sample is a plasma sample, t2 is chosen to be 600 seconds after t1.

6. (canceled)

7. Method according to claim 1, wherein the patient:
i. may have or has a deficiency in at least one coagulation factor chosen among: factor VIII, factor IX, factor XI, in particular is diagnosed as having hemophilia A, hemophilia B, or hemophilia C, and/or
ii. is being treated by supplementation with a coagulation factor chosen among: factor VIII, factor IX, factor XI, factor XIII, and/or
iii. is being treated with bispecific antibody treatment, or
iv. is being treated by what is referred to as “bypassing” therapy (for example, NovoSeven®), or
v. is being treated with a therapy targeting both hemophilia A and hemophilia B, for example such as Fitusiran® (Sanofi) or Concizumab® (NovoNordisk)
vi. is being treated with antifibrinolytic therapy, for example tranexamic acid.

8. Method according to claim 1, wherein, in step d., implementation of the degradation kinetics of the fibrin clot due to its lysis is carried out by a measurement method in particular chosen among: a viscoelastic method, a rheometric method, an acoustic method, an optical method, a waveform analysis method, a fluorometric method, a magnetic resonance method, a turbidimetric method.

9. Method according to claim 1, wherein the degradation kinetics of the fibrin clot in step d. are carried out by turbidimetry, in particular by measurement of the optical density (OD) at a wavelength between 350 and 800 nm, preferably at 540 nm, and for a duration of between 1400 to 3600 seconds starting from the triggering of coagulation by the addition of calcium ions, and wherein the time t1 is chosen to be within the range of 700 to 900 seconds after coagulation is triggered by the addition of calcium ions, and the time t2 is chosen to be within the range of 1100 to 1400 seconds after coagulation is triggered by the addition of calcium ions.

10. (canceled)

11. Method according to claim 1, wherein the degradation kinetics of the fibrin clot in step d. are carried out by a viscoelastic measurement method, and for a duration of between 1400 and 3600 seconds starting from the triggering of coagulation by the addition of calcium ions, and wherein time t1 is chosen to be within the range of 900 to 1200 seconds after coagulation is triggered by the addition of calcium ions and time t2 is chosen to be within the range of 1500 to 1800 seconds after coagulation is triggered by the addition of calcium ions.

12. (canceled)

13. Method according to claim 1, comprising an additional step f. of classifying the tested sample into a group reflecting a lysis profile of a fibrin clot, said lysis profile being determined on the basis of the values measured at t1 and t2 in step e., the classification being made into a group reflecting a lysis profile of a fibrin clot, in particular into three distinct groups.

14-15. (canceled)

16. Method according to claim 1, comprising an additional step f. of classifying the tested sample into a group reflecting a lysis profile of a fibrin clot, said lysis profile being determined on the basis of the values measured at t1 and t2 in step e., the classification being made into a group reflecting a lysis profile of a fibrin clot, in particular into three distinct groups and wherein the classification is carried out via a classification model that is predefined on the basis of classification parameters obtained with training data processed under the same experimental conditions as those of the analyzed sample, the classification being made on the basis of the values measured at t1 and t2 in step e. described in claim 1.

17. Method according to claim 16, wherein the classification of the tested sample into a group reflecting a lysis profile of a fibrin clot is carried out on the basis of a classification model obtained by unsupervised or supervised learning.

18. Method according to claim 17, wherein the classification model is obtained by a learning method chosen among: a hierarchical analysis, a K-means method, a quadratic discriminant analysis, logistic regression, or a random forest method.

19. Method for monitoring a therapeutic treatment administered to a patient who may have or who does have a deficiency in at least one coagulation factor, comprising the following steps:
a. Implementing a method for performing an in vitro measurement of a curve of the lysis of a fibrin clot over time according to claim 1, on a blood or plasma sample obtained from said patient, at at least one given time and possibly at another or several subsequent times, said method including a step f. of classifying the tested sample into a group reflecting a lysis profile of a fibrin clot;
b. On the basis of the classification into a group that was obtained for the analyzed sample, making a conclusion concerning the deficiency in coagulation factor(s) of the analyzed patient observed by the classification method or concerning the state of health of said patient.

20. Method according to claim 19, further comprising a step of adjusting the therapeutic treatment followed by said patient, according to the classification obtained.

21. Method for treating a patient who may have or who does have a deficiency in at least one coagulation factor, comprising implementing a method according to claim 1 on a sample of said patient in need thereof, for classification of the sample, monitoring, or making an adjustment to the therapeutic treatment of said patient, and administering to said patient an antifibrinolytic agent, in particular tranexamic acid (TXA) or a composition comprising an antifibrinolytic agent or tranexamic acid (TXA).

22. Method for screening a therapeutic molecule or a treatment against hemophilia in order to determine whether said molecule or said therapeutic treatment allows modifying the lysis profile of a fibrin clot, comprising the following steps:
a. Implementing a method for performing an in vitro measurement of a curve of the lysis of a fibrin clot over time according to claim 1, on a blood or plasma sample previously obtained from said patient at a given time, and
b. Implementing a method for performing an in vitro measurement of a curve of the lysis of a fibrin clot over time according to claim 1, on a blood or plasma sample previously obtained from said patient at a given time that is different from the time in step a., after administering to said patient a therapeutic molecule or treatment aimed at modifying his or her lysis profile of a fibrin clot and where appropriate also treating a hemophilia condition, and
c. Comparing the curves of the lysis of a fibrin clot over time which are obtained in steps a. and b.,
d. Making a conclusion concerning the ability of the therapeutic molecule or treatment to modify the lysis profile of a fibrin clot, after comparing the curves of the lysis of a fibrin clot over time obtained in steps a. and b.

23. Data processing system or device comprising means for implementing at least step e. of claim 1, in order to return the classification result which takes into account the variables provided as input.

24. (canceled)

25. Non-transitory computer-readable storage medium on which is stored a program for implementing the method according to claim 1 when this program is executed by a processor.

26. Kit, in particular adapted for implementing a method according to claim 1, comprising:
a. one or more of the following reagents: tissue factor, phospholipids, tPA, an activator of the intrinsic coagulation pathway chosen among: ellagic acid, ethylene glycol gallate, silica, FIXa, FXIa, FXIIa, or several of these, calcium ions,
b. Optionally, a coagulation factor chosen among factor VIII, factor IX, factor XI, or several of these,
c. Optionally, bispecific antibodies, for example emicizumab or Hemlibra® (Roche), or several of these,
d. Optionally, one or more appropriate buffers,
e. Optionally, instructions for carrying out one or more degradation kinetics of a fibrin clot, and
f. Optionally, a system and/or device comprising means for implementing at least step e. of claim 1, in order to return the classification result which takes into account the variables provided as input and/or a non-transitory computer-readable storage medium on which is stored a program for implementing the method according to claim 1 when this program is executed by a processor,
g. Optionally, instructions which allow implementing the method according to claim 1,
h. Optionally, instructions relating to the use of a signal from a data medium, for the implementation of a method according to claim 1.
